(19) United States
(12) Patent Application Publication
(10) Pub. No.: US 2010/0077498 A1
(43) Pub. Date: Mar. 25, 2010

(54) COMPOSITIONS AND METHODS FOR BLOOD-BRAIN BARRIER DELIVERY IN THE MOUSE

(76) Inventors: William M. Pardridge, Pacific Palisades, CA (US); Ruben J. Boado, Agoura Hills, CA (US)

Correspondence Address: WILSON, SONSINI, GOODRICH & ROSATI, 650 PAGE MILL ROAD, PALO ALTO, CA, 94304-1050, US

(21) Appl. No.: 12/558,348

(22) Filed: Sep. 11, 2009

Related U.S. Application Data
(60) Provisional application No. 61/096,111, filed on Sep. 11, 2008.

(51) Int. Cl. A61K 39/395; C07K 16/00; C07H 21/04; A01K 67/027; A61P 25/00

(52) U.S. Cl. 800/18; 530/387.3; 424/133.1; 536/23.53

(57) ABSTRACT

The invention provides compositions and methods, for increasing transport of CNS-active agents across the blood brain barrier in a mouse, e.g., a mouse model of a human CNS condition, while allowing their activity once across the barrier to remain substantially intact. The CNS-active agents are transported across the blood brain barrier via the mouse transferrin receptor. In some embodiments the agents are therapeutic, diagnostic, or research agents.

### CROSS-REFERENCE

This application claims the benefit of U.S. Provisional Patent Application No. 61/096,111, entitled “Compositions and Methods for Blood-Brain Barrier Delivery in the Mouse,” filed on Sep. 11, 2008, the contents of which are herein incorporated by reference in their entirety.

### BACKGROUND OF THE INVENTION

Biopharmaceuticals, such as recombinant proteins, monoclonal antibodies, or short interfering RNA, generally do not cross the blood-brain barrier (BBB). However, biopharmaceuticals can be delivered to the brain, across the BBB, with “molecular Trojan horse” technology. In this approach, a fusion protein is engineered in which the therapeutic protein is fused to a protein, e.g., a chimeric monoclonal antibody that crosses the BBB via receptor-mediated transport (RMT) on an endogenous transporter at the BBB (e.g., an insulin receptor). Generally, the molecular trojan horses that have been developed are specific for human transporter systems. However, there is a need for a mouse-specific molecular Trojan horse, which can be used to generate fusion proteins for pre-clinical testing of both efficacy and toxicity in the mouse of therapeutic fusion proteins that are being developed as human neuropharmaceuticals.

### SUMMARY OF THE INVENTION

The present invention provides compositions and methods for delivering a CNS-active agent across the BBB in a mouse. Accordingly, on one aspect provided herein is a composition comprising a purified chimeric monoclonal antibody against the mouse transferrin receptor. In some embodiments, the composition comprises a fusion protein comprising the chimeric monoclonal antibody against the mouse transferrin receptor and a CNS-active polypeptide (e.g., a neurotrophin, a single chain Fv antibody, or an avidin), where the CNS-active polypeptide is covalently linked to either the heavy chain or the light chain of the chimeric monoclonal antibody. In some embodiments, the chimeric monoclonal antibody and the CNS-active polypeptide in the fusion protein each retain an average of at least 10% (e.g., at least about 20%, 30%, 40%, 50%, 60%, 70%, 80%, 90%, 95%, 99%, or 100%) of their activities as separate entities. In some embodiments, the CNS active polypeptide comprises the amino acid sequence of a neurotrophin, a single chain Fv antibody, an avidin, or an enzyme. In some embodiments, the CNS active polypeptide is covalently linked at its N-terminus to the C-terminus of the chimeric monoclonal antibody heavy chain or light chain. In some embodiments, a therapeutic agent is delivered across the BBB in a mouse by administering any of the foregoing compositions to the mouse.

In another aspect provided herein is a nucleic acid encoding a heavy chain immunoglobulin or a light chain immunoglobulin of a monoclonal antibody (e.g., a monoclonal antibody) against the mouse transferrin receptor. In some embodiments, the nucleic acid further encodes a CNS-active polypeptide fused in frame to the encoded heavy chain immunoglobulin or light chain immunoglobulin. In some embodiments, the encoded CNS-active polypeptide comprises the amino acid sequence of a neurotrophin, a single chain Fv antibody, an avidin, or an enzyme. In some embodiments, the nucleic acid hybridizes under medium stringency (or high stringency) conditions to a nucleic acid comprising the nucleic acid sequence of any of SEQ ID NOs: 13, 16, 20, or its complement. In some embodiments, the nucleic acid hybridizes under medium stringency (or high stringency) conditions to a nucleic acid encoding a polypeptide comprising the amino acid sequence of any of SEQ ID NOs:14, 15, 17, 19, 21, or to the complement of the nucleic acid sequence encoding the polypeptide.

In a further aspect provided herein is a recombinant mouse comprising a chimeric monoclonal antibody against the mouse transferrin receptor. In some embodiments, the recombinant mouse comprises a fusion protein comprising the chimeric monoclonal antibody against the mouse transferrin receptor and a CNS-active polypeptide, where the CNS-active polypeptide is covalently linked to a heavy chain or a light chain of the chimeric monoclonal antibody. In some embodiments, the CNS-active polypeptide comprises the amino acid sequence of a neurotrophin, a single chain Fv antibody, an avidin, or an enzyme. In some embodiments, the CNS-active polypeptide is covalently linked at its N-terminus to the C-terminus of the chimeric monoclonal antibody heavy chain or light chain. In some embodiments, the CNS-active polypeptide comprises an amino acid sequence at least 85% identical to that of a human neurotrophin.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Agarose gel electrophoresis and ethidium bromide staining of PCR cloning of 0.4 kb TfRMAb VH (A), 0.4 kb TfRMAb VL (B), 1.4 kb mouse IgG1 C-region (C), and 0.7 kb mouse kappa C-region (D). The PCR generated cDNA is shown in lane 1, and DNA size standards are shown in lanes 2 and 3 for each panel.

FIG. 2. Genetic engineering of the eukaryotic heavy chain (HC) expression plasmid, pCD-HC, and the light chain (LC) expression plasmid, pCD-LC, is shown in Panels A and B, respectively. The variable region of the HC (VH) of the chimeric TfRMAb is fused to the C-region of mouse IgG1 (mIgG1) in pCD-HC, and the variable region of the LC (VL) of the chimeric TfRMAb is fused to the C-region of mouse kappa (mKappa) in pCD-LC.

FIG. 3. Deduced amino acid sequence of the chimeric TfRMAb heavy chain (A) and light chain (B). The individual complementarity determining regions (CDR) and framework regions (FR) of the VH and VL are shown. The HC C-region is comprised of 4 sub-domains: CH1, hinge, CH2, and CH3. The LC C-region is denoted as CL.

FIG. 4. Western blot shows identical reactivity with an anti-mouse antibody of the chimeric TfRMAb (lane 1) and the 8D3 rat hybridoma-generated TfRMAb (lane 2).

FIG. 5. Radio-receptor assay of the mouse TfR uses mouse fibroblasts as the source of the mouse TfR and [¹²⁵I]-8D3 as the binding ligand. Binding is displaced by unlabeled 8D3 MAb or the chimeric TfRMAb. The KD of 8D3 self-inhibition and the KI of chimeric TfRMAb cross-inhibition were computed by non-linear regression analysis.

FIG. 6. Genetic engineering of tandem vector (TV) encoding the chimeric TfRMAb heavy chain (HC) and light chain (LC) from 3 precursor plasmids: pCD-HC, pCD-LC, and pwtDWHFR. The engineering of the pCD-HC and pCD-LC plasmids is outlined in FIG. 2. The pwtCHFR encodes for the wild type (wt) murine dihydrofolate reductase (DHFR). The HC and LC expression cassettes have the cytomegalovirus (CMV) promoter at the 5′-end and the bovine growth hormone (BGH) polyA+ sequence at the 3′-end. The DHFR expression cassette has the SV50 promoter at the 5′-end and the hepatitis B virus polyA+ sequence at the 3′-end. Amp=ampicillin resistance gene; Neo=neomycin resistance gene; ori=origin of replication. The HC gene contains a unique HpaI restriction endonuclease sequence at the most 5′ end of the open reading frame, which allows for insertion of the cDNA encoding the therapeutic protein at this site.

FIG. 7. Tandem vector encoding separate and tandem expression cassettes producing the fusion protein of the chimeric (c) TfRMAb heavy chain, fused to human glial derived neurotrophic factor (GDNF), light chain of the chimeric TfRMAb, and the murine dihydrofolate reductase (DHFR).

FIG. 8. Structure of cTfRMAb-GDNF fusion protein, where human GDNF is fused to the carboxyl terminus of the heavy chain of the chimeric MAb against the mouse TfR.

FIG. 9. Western blot of cTfRMAb-GDNF fusion protein or GDNF with primary antibodies against human GDNF (left panel) or mouse IgG (right panel).

FIG. 10. (A) Outline of GFRα1 receptor binding assay. The GFRα1:Fc fusion protein is captured by a mouse anti-human (MAH) Fc antibody. The GDNF, or cTfRMAb-GDNF fusion protein, binds to the GFRα1, and this binding is detected with a goat anti-GDNF antibody and a rabbit anti-goat (RAG) antibody conjugated to alkaline phosphatase (AP). (B) Binding of either GDNF (top panel) or the cTfRMAb-GDNF fusion protein (bottom panel) to the GFRα1 extracellular domain (ECD) is saturable. The ED50 of the cTfRMAb-GDNF binding to the GFRα1 ECD is comparable to the ED50 of the binding of recombinant GDNF.

FIG. 11. Radio-receptor assay of the mouse TfR uses mouse fibroblasts as the source of the mouse TfR and [¹²⁵]-8D3 as the binding ligand. Binding is displaced by unlabeled 8D3 MAb (left panel) or the cTfRMAb-GDNF fusion protein (right panel). The KD of 8D3 self-inhibition and the KI of cTfRMAb-GDNF fusion protein cross-inhibition were computed by non-linear regression analysis. There is no significant difference in affinity of the cTfRMAb to the mouse TfR following fusion of the GDNF to the antibody.

FIG. 12. Radio-receptor assay of the mouse TfR uses mouse fibroblasts as the source of the mouse TfR and [¹²⁵]-8D3 as the binding ligand. Binding is displaced by unlabeled 8D3 MAb or the cTfRMAb-avidin fusion protein. The KD of 8D3 self-inhibition and the KI of the cTfRMAb-avidin cross-inhibition were computed by non-linear regression analysis. There is no significant difference in affinity of the cTfRMAb to the mouse TfR following fusion of the avidin to the antibody.

FIG. 13. (A) Plasma concentration of [¹²⁵I]-cTfRMAb in the mouse is expressed as % of injected dose (ID)/mL, and is plotted vs time after a single intravenous injection in the anesthetized mouse. (B) Plasma radioactivity that is precipitable by trichoroacetic acid (TCA) is plotted vs. time after intravenous injection.

FIG. 14. The organ volume of distribution (VD) in the mouse at 60 min after intravenous injection is shown for brain, heart, liver, and kidney for the [¹²⁵I]-cTfRMAb (open bars), the [¹²⁵I]-OX26 TfRMAb (solid bars), and the [¹²⁵I]-8D3 TfRMAb (gray bars).

### DETAILED DESCRIPTION OF THE INVENTION

### Table of Contents

### I. Introduction

### II. Definitions

### III. The Blood Brain Barrier

IV. Exemplary Agents for transport across the mouse blood brain barrier

A. Neurotrophins

B. Antibodies

C. Avidin Conjugates

D. Enzymes

### V. Compositions

VI. Nucleic acids, vectors, cells, and manufacture

A. Nucleic acids

B. Vectors

C. Cells

D. Manufacture

### VII. Recombinant Mice

### VIII. Methods

### IX. Examples

### X. Sequences and SEQ ID NOs

### ABBREVIATIONS

AA amino acid
AD Alzheimer's disease
AP alkaline phosphatase
BBB blood-brain barrier
BCA bicinchoninic acid
BGH bovine growth hormone
CDR complementarity determining region
CHO Chinese hamster ovary
CMV cytomegalovirus
DC dilutional cloning
DHFR dihydrofolate reductase
ECD extracellular domain
ED50 effective dose causing 50% saturation
FR framework region
FS flanking sequence
FWD forward
GDNF glial derived neurotrophic factor
GFR GDNF receptor
HC heavy chain
TfRMAb HC heavy chain of TfRMAb
TfRMAb LC light chain of TfRMAb
HPLC high pressure liquid chromatography
HT hypoxanthine-thymidine
ID injected dose
IgG immunoglobulin G
LC light chain
MAb monoclonal antibody
MAH mouse anti-human IgG
MTX methotrexate
MW molecular weight
N asparagine
nt nucleotide
ODN oligodeoxynucleotide
orf open reading frame
pA poly-adenylation
PAGE polyacrylamide gel electrophoresis
PBS phosphate buffered saline
PBST PBS plus Tween-20
PCR polymerase chain reaction
PD Parkinson's disease
PVDF Polyvinylidene fluoride
R receptor
REV reverse
RMT receptor-mediated transport
RNase A ribonuclease A
RT reverse transcriptase
RT room temperature
ScFv single chain Fv antibody
SDM site-directed mutagenesis
SDS sodium dodecyl sulfate
SFM serum free medium
TH Trojan horse\
TfR transferrin receptor
TfRMAb MAb against the TfR
cTfRMAb chimeric MAb against the mouse TfR
cTrFMAb-GDNF fusion protein of GDNF and the chimeric TfRMAb
TV tandem vector
UTV universal TV
VH variable region of heavy chain
VL variable region of light chain

### I. Introduction

The blood brain barrier is a limiting factor in the delivery of many peripherally-administered agents to the central nervous system of a mouse, e.g., a transgenic disease model mouse. The present invention addresses three factors that are important in delivering an agent across the BBB to the CNS: 1) A pharmacokinetic profile for the agent that allows sufficient time in the peripheral circulation for the agent to have enough contact with the BBB to traverse it; 2) Modification of the agent to allow it to cross the BBB; and 3) Retention of activity of the agent once across the BBB. Various aspects of the invention address these factors, by providing fusion structures (e.g., fusion proteins) of an agent (e.g., a therapeutic agent) covalently linked to a monoclonal antibody against the mouse transferrin receptor, (mouse TfRMAb) and is transported across the BBB, and/or to retain some or all of its activity in the brain while still attached to the structure.

Accordingly, in one aspect, the invention provides compositions and methods that utilize an agent covalently linked to a mouse TfRMAb for delivery across the BBB into the CNS. The compositions and methods are useful in transporting agents, e.g., therapeutic agents such as neurotherapeutic agents, from the peripheral blood and across the BBB into the CNS. Neurotherapeutic agents useful in the invention include, but are not limited to, neurotrophins, e.g. Glial-Derived Neurotrophic Factor (GDNF); ScFv antibodies (e.g., anti-Aβ peptide antibodies), and avidin-biotin conjugates (e.g., conjugates of avidin and biotinylated nucleic acids). In some embodiments, the mouse TfRMAb that crosses the BBB is a chimeric MAb, i.e., a cTfRMAb.

In some embodiments, the invention provides a fusion protein that includes a mouse TfRMAb covalently linked to a CNS-active polypeptide (CNS), where the TfRMAb and the CNS-active polypeptide, or a CNS-active polypeptide conjugate in the central nervous system each retain a proportion (e.g., 10-100%) of their activities (or their binding affinities for their respective receptors), compared to their activities (e.g., binding affinities) as separate entities.

The invention also provides nucleic acids encoding fusion proteins. In some embodiments, the invention provides a single nucleic acid sequence that contains a gene coding for a light chain of a mouse TfRMAb immunoglobulin and/or a gene coding for a fusion protein made up of a heavy chain of a mouse TfRMAb immunoglobulin covalently linked to a CNS-active polypeptide. In some embodiments the polypeptide of the fusion protein is a therapeutic polypeptide, e.g., a neurotherapeutic polypeptide such as a neurotrophin. The invention also provides vectors containing the nucleic acids of the invention, and cells containing the vectors. Further provided are methods of manufacturing an immunoglobulin fusion protein, where the fusion protein contains an immunoglobulin heavy chain fused to a therapeutic agent, where the methods include permanently integrating into a eukaryotic cell a single tandem expression vector in which both the immunoglobulin light chain gene and the gene for the immunoglobulin heavy chain fused to the CNS-active polypeptide are incorporated into a single piece of DNA.

The invention further provides therapeutic compositions, such as pharmaceutical compositions that contain a CNS-active polypeptide covalently linked to a mouse TfRMAb and a pharmaceutically acceptable excipient. In some embodiments, the invention provides a composition for delivering a nucleic acid to the CNS of a mouse, which includes an avidin-biotinylated nucleic acid conjugate covalently linked to a mouse TfRMAb.

The invention also provides methods for treating a neurological disorder in a mouse disease model that include peripherally administering to the mouse a dose of one or more of the compositions of the invention, optionally in combination with other therapy for the disorder.

### II. Definitions

As used herein, an “agent” includes any substance that is useful in producing an effect, including a physiological or biochemical effect in an organism. A “therapeutic agent” is a substance that produces or is intended to produce a therapeutic effect, i.e., an effect that leads to amelioration, prevention, retarded progression, and/or complete or partial cure of a disorder. A “therapeutic effect,” as that term is used herein, also includes the production of a condition that is better than the average or normal condition in an individual that is not suffering from a disorder, i.e., a supranormal effect such as improved cognition, memory, mood, or other characteristic attributable at least in part to the functioning of the CNS, compared to the normal or average state. A “neurotherapeutic agent” is an agent that produces a therapeutic effect in the CNS. A “therapeutic polypeptide” includes therapeutic agents that consists of a polypeptide. A “cationic therapeutic polypeptide” encompasses therapeutic polypeptides whose isoelectric point is above about 7.4, in some embodiments, above about 8, 8.5, 9, 9.5, 10, 10.5, 11, 11.5, 12, or above about 12.5. A subcategory of cationic therapeutic polypeptides is cationic neurotherapeutic polypeptides.

As used herein, a “central nervous system (CNS)-active agent” is an agent that has an effect when delivered to the CNS. For example, a “central nervous system (CNS)-active polypeptide” includes peptides, polypeptides, and proteins that have an effect when administered to the CNS. The effect may be a therapeutic effect or a non-therapeutic effect, e.g., a diagnostic effect or an effect useful in research. If the effect is a therapeutic effect, then the polypeptide is also a therapeutic peptide. A therapeutic polypeptide that is also a polypeptide that is active in the CNS is encompassed by the term “neurotherapeutic polypeptide,” as used herein. A CNS-active polypeptide may act directly or indirectly in the CNS. A non-limiting example of a CNS-active polypeptide that acts directly is a neurotrophin (e.g., BDNF). A non-limiting example of a CNS-active polypeptide that acts indirectly is avidin, which may bind to a biotinylated agent (e.g., siRNA) that acts directly in the CNS. The term CNS-active agent, as used herein, also encompasses, non-covalent complexes of a mouse TfRMAb fusion protein with a non-peptide therapeutic agent, e.g., a nucleic acid, or a small molecule compound that requires delivery across the BBB.

“Treatment” or “treating” as used herein includes achieving a therapeutic benefit and/or a prophylactic benefit. By therapeutic benefit is meant eradication or amelioration of the underlying disorder or condition being treated. For example, in an individual with a neurological disorder, therapeutic benefit includes partial or complete halting of the progression of the disorder, or partial or complete reversal of the disorder. Also, a therapeutic benefit is achieved with the eradication or amelioration of one or more of the physiological or psychological symptoms associated with the underlying condition such that an improvement is observed in the patient, notwithstanding the fact that the patient may still be affected by the condition. A prophylactic benefit of treatment includes prevention of a condition, retarding the progress of a condition (e.g., slowing the progression of a neurological disorder), or decreasing the likelihood of occurrence of a condition. As used herein, “treating” or “treatment” includes prophylaxis.

As used herein, the term “effective amount” can be an amount sufficient to effect beneficial or desired results, such as beneficial or desired clinical results, or enhanced cognition, memory, mood, or other desired CNS results. An effective amount is also an amount that produces a prophylactic effect, e.g., an amount that delays, reduces, or eliminates the appearance of a pathological or undesired condition. Such conditions of the CNS include dementia, neurodegenerative diseases as described herein, suboptimal memory or cognition, mood disorders, general CNS aging, or other undesirable conditions. An effective amount can be administered in one or more administrations. In terms of treatment, an “effective amount” of a composition of the invention is an amount that is sufficient to palliate, ameliorate, stabilize, reverse or slow the progression of a disorder, e.g., a neurological disorder. An “effective amount” may be of any of the compositions of the invention used alone or in conjunction with one or more agents used to treat a disease or disorder. An “effective amount” of a therapeutic agent within the meaning of the present invention can be determined by a patient's attending physician or veterinarian. Such amounts are readily ascertained by one of ordinary skill in the art and will a therapeutic effect when administered in accordance with the present invention. Factors which influence what a therapeutically effective amount will be include, the specific activity of the therapeutic agent being used, the type of disorder (e.g., acute vs. chronic neurological disorder), time elapsed since the initiation of the disorder, and the age, physical condition, existence of other disease states, and nutritional status of the individual being treated. Additionally, other medication the patient may be receiving will affect the determination of the therapeutically effective amount of the therapeutic agent to administer.

A “subject” or an “individual,” as used herein, is a rodent. In some embodiments, the rodent is a mouse. In some embodiments, the subject is a mouse that is suffering from an experimentally induced neurological disorder.

In some embodiments, an agent is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, subcutaneous, intramuscular, intraperitoneal, transdermal, inhalation, transbuccal, intranasal, rectal, and oral administration.

A “pharmaceutically acceptable carrier” or “pharmaceutically acceptable excipient” herein refers to any carrier that does not itself induce the production of antibodies harmful to the individual receiving the composition. Such carriers are well known to those of ordinary skill in the art. A thorough discussion of pharmaceutically acceptable carriers/excipients can be found in Remington's Pharmaceutical Sciences, Gennaro, Ariz., ed., 20th edition, 2000: Williams and Wilkins Pa., USA. Exemplary pharmaceutically acceptable carriers can include salts, for example, mineral acid salts such as hydrochlorides, hydrobromides, phosphates, sulfates, and the like; and the salts of organic acids such as acetates, propionates, malonates, benzoates, and the like. For example, compositions of the invention may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate.

A “recombinant host cell” or “host cell” refers to a cell that includes an exogenous polynucleotide, regardless of the method used for insertion, for example, direct uptake, transduction, transfection, f-mating, or other methods known in the art to create recombinant host cells. The exogenous polynucleotide may be maintained as a nonintegrated vector, for example, a plasmid, or alternatively, may be integrated into the host genome.

A “recombinant mouse,” as used herein, refers to any mouse into which an exogenous nucleic acid or polypeptide has been introduced. In one non-limiting example, a recombinant mouse is a mouse that has been administered a fusion protein comprising a mouse TfRMAb covalently linked to a CNS-active polypeptide. In another non-limiting example, a recombinant mouse is a mouse that has been administered a TfRMAb-Avidin fusion protein complexed (non-covalently) with an siRNA. In another non-limiting example, a recombinant mouse is a mouse that has been administered an autologous or heterologous cell genetically modified to secrete a mouse TfRMAb fusion antibody.

The terms “polypeptide,” “peptide” and “protein” are used interchangeably herein to refer to a polymer of amino acid residues. That is, a description directed to a polypeptide applies equally to a description of a polypeptide and a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent polypeptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified polypeptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid may be one that is separated from the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and peptides are well known in the art.

### III. The blood brain barrier

In one aspect, the invention provides compositions and methods that utilize a CNS-active polypeptide covalently linked to a mouse TfRMAb. The compositions and methods are useful in transporting agents, e.g. therapeutic agents such as neurotherapeutic agents, from the peripheral blood and across the blood brain barrier into the CNS. As used herein, the “blood-brain barrier” refers to the barrier between the peripheral circulation and the brain and spinal cord which is formed by tight junctions within the brain capillary endothelial plasma membranes, creates an extremely tight barrier that restricts the transport of molecules into the brain, even molecules as small as urea, molecular weight of 60 Da. The blood-brain barrier within the brain, the blood-spinal cord barrier within the spinal cord, and the blood-retinal barrier within the retina, are contiguous capillary barriers within the central nervous system (CNS), and are collectively referred to as the blood-brain barrier or BBB.

Delivery across the BBB is a limiting step in the development of new neurotherapeutics, diagnostics, and research tools for the brain and CNS. Essentially 100% of large molecule therapeutics such as recombinant proteins, antisense drugs, gene medicines, monoclonal antibodies, or RNA interference (RNAi)/siRNA-based drugs, do not cross the BBB in pharmacologically significant amounts. While it is generally assumed that small molecule drugs can cross the BBB, in fact, <2% of all small molecule drugs are active in the brain owing to the lack of transport across the BBB. A molecule must be lipid soluble and have a molecular weight less than 400 Daltons (Da) in order to cross the BBB in pharmacologically significant amounts, and the vast majority of small molecules do not have these dual molecular characteristics. Therefore, most potentially therapeutic, diagnostic, or research molecules do not cross the BBB in pharmacologically active amounts. So as to bypass the BBB, invasive transcranial drug delivery strategies are used, such as intracerebro-ventricular (ICV) infusion, intracerebral (IC) administration, and convection enhanced diffusion (CED). Transcranial drug delivery to the brain is expensive, invasive, and largely ineffective. The ICV route delivers BDNF only to the ependymal surface of the brain, not into brain parenchyma, which is typical for drugs given by the ICV route. The IC administration of a neurotrophin, such as nerve growth factor (NGF), only delivers drug to the local injection site, owing to the low efficiency of drug diffusion within the brain. The CED of neurotrophin results in preferential fluid flow through the white matter tracts of brain, which causes demyelination, and astrogliosis.

The present invention offers an alternative to these highly invasive and generally unsatisfactory methods for bypassing the BBB, allowing agents, e.g., neuroprotective factors, to cross the BBB from the peripheral blood. It is based on the use of endogenous transport systems present in the BBB to provide a mechanism to transport a desired substance from the peripheral blood to the CNS.

In some embodiments, the invention provides compositions that include, a mAb against the mouse transferrin receptor mediated transport system coupled to a CNS-active agent for which transport across the BBB is desired, e.g., a neurotherapeutic agent. In some embodiments, the mouse TfR monoclonal antibody is a chimeric antibody (cTfRMAb), e.g., a rat-mouse chimeric antibody. In other embodiments, the antibody is 100% murinized. In some embodiments, the antibody is a monoclonal antibody (MAb), e.g., a cTfRMAb. Generally, the TfRMAbs are directed to the extracellular domain of the mouse TfR. In one embodiment, the TfRMAb comprises the CDRs of the rat 8D3 MAb against the mouse TfR as described in Lee et al (2000), J. Pharmacol. Exp. Ther., 292: 1048-1052.

An “antibody,” as used herein, includes reference to any molecule, whether naturally-occurring, artificially induced, or recombinant, which has specific immunoreactive activity. Generally, though not necessarily, an antibody is a protein that includes two molecules, each molecule having two different polypeptides, the shorter of which functions as the light chains of the antibody and the longer of which polypeptides function as the heavy chains of the antibody. Normally, as used herein, an antibody will include at least one variable region from a heavy or light chain. Additionally, the antibody may comprise combinations of variable regions. The combination may include more than one variable region of a light chain or of a heavy chain. The antibody may also include variable regions from one or more light chains in combination with variable regions of one or more heavy chains. An antibody can be an immunoglobulin molecule obtained by in vitro or in vivo generation of the humoral response, and includes both polyclonal and monoclonal antibodies. An antibody also may be obtained via recombinant DNA techniques, e.g., by using host cells transformed with heavy and/or light chain genes. Furthermore, the present invention includes antigen binding fragments of the antibodies described herein, such as Fab, Fab′, F(ab)₂, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. Such antibody fragments may be produced by chemical methods, e.g., by cleaving an intact antibody with a protease, such as pepsin or papain, or via recombinant DNA techniques, e.g., by using host cells transformed with truncated heavy and/or light chain genes. Synthetic methods of generating such fragments are also contemplated. Heavy and light chain monomers may similarly be produced by treating an intact antibody with a reducing agent, such as dithiothreitol or .beta.-mercaptoethanol, or by using host cells transformed with DNA encoding either the desired heavy chain or light chain or both. An antibody immunologically reactive with a particular antigen can be generated in vivo or by recombinant methods such as selection of libraries of recombinant antibodies in phage or similar vectors.

A “chimeric” antibody includes an antibody derived from a combination of different mammals. The mammal may be, for example, a rabbit, a mouse, a rat, or a goat. The combination of different mammals includes combinations of fragments from rat and mouse sources.

In some embodiments, an antibody of the present invention is a monoclonal antibody (MAb), typically a rat monoclonal antibody.

For use in mice, a chimeric MAb is preferred that contains enough mouse sequence that it is not significantly immunogenic when administered to mice, e.g., about 80% mouse and about 20% rat, or about 85% mouse and about 15% rat, or about 90% mouse and about 10% rat, or about 95% mouse and 5% rat, or greater than about 95% mouse and less than about 5% rat.

Antibodies used in the invention may be glycosylated or non-glycosylated. If the antibody is glycosylated, any pattern of glycosylation that does not significantly affect the function of the antibody may be used. Glycosylation can occur in the pattern typical of the cell in which the antibody is made, and may vary from cell type to cell type. For example, the glycosylation pattern of a monoclonal antibody produced by a mouse myeloma cell can be different than the glycosylation pattern of a monoclonal antibody produced by a transfected Chinese hamster ovary (CHO) cell. In some embodiments, the antibody is glycosylated in the pattern produced by a transfected Chinese hamster ovary (CHO) cell.

Accordingly, in some embodiments, a genetically engineered mouse TfRMAb, with the desired level of mouse sequences, is fused to a CNS-active polypeptide for which transport across the BBB is desired, e.g. a neurotherapeutic agent such as a neurotrophin such as GDNF, to produce a recombinant fusion protein that is a bi-functional molecule. The recombinant therapeutic neuroprotective factor/mouse TfRMAb is able to both (i) cross the mouse BBB, via transport on the BBB TfR, and (ii) activate the factor's target, e.g., the GDNF receptor, to cause neurotherapeutic effects once inside the brain, following peripheral administration.

### IV. Exemplary Agents for Transport Across the BBB

The agent for which transport across the BBB is desired may be any suitable substance for introduction into the CNS. Generally, the agent is a substance for which transport across the BBB is desired, which does not, in its native form, cross the BBB in significant amounts. The agent may be, e.g., a therapeutic agent, a diagnostic agent, or a research agent. Diagnostic agents include polypeptide radiopharmaceuticals, such as the epidermal growth factor (EGF) for imaging brain cancer (Kurihara and Pardridge (1999) Canc. Res. 54: 6159-6163), and amyloid peptides for imaging brain amyloid such as in Alzheimers disease (Lee et al (2002) J. Cereb. Blood Flow Metabol. 22: 223-231). In some embodiments, the agent is a therapeutic agent, such as a neurotherapeutic agent. Apart from neurotrophins, potentially useful therapeutic protein agents include recombinant enzymes for lysosomal storage disorders (see, e.g., U.S. Patent Application Publication No. 20050142141, filed Feb. 17, 2005, incorporated by reference herein in its entirety), monoclonal antibodies that either mimic an endogenous polypeptide or block the action of an endogenous peptide, polypeptides for brain disorders, such as secretin for autism (Ratliff-Schaub et al (2005) Autism 9: 256-265), opioid peptides for drug or alcohol addiction (Cowen et al, (2004) J. Neurochem. 89: 273-285), or neuropeptides for appetite control (Jethwa et al (2005) Am. J. Physiol. 289: E301-305). In some embodiments, the agent is a neurotrophic factor, also referred to herein as a “neurotrophin.” Thus, in some embodiments, the invention provides compositions and methods that utilize a neurotrophin. In some embodiments, a single neurotrophin may be used. In others, combinations of neurotrophins are used. In some embodiments, the invention utilizes a glial-derived neurotrophic factor (GDNF).

A. Neurotrophins

Many neurotrophic factors are neuroprotective in brain, but do not cross the blood-brain barrier. These factors are suitable for use in the compositions and methods of the invention and include glial-derived neurotrophic factor (GDNF), brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-α, TGF-β, vascular endothelial growth factor (VEGF), interleukin-1 receptor antagonist (IL-1ra), ciliary neurotrophic factor (CNTF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, and stem cell factor (SCF). Particularly useful in some embodiments of the invention utilizing neurotrophins that are used as precursors for fusion proteins that cross the BBB are those that naturally form dimeric structures, similar to BDNF. Certain neurotrophins such as BDNF or NT-3 may form hetero-dimeric structures, and in some embodiments the invention provides a fusion protein constructed of one neurotrophin monomer fused to one chain (e.g., a light or heavy chain) of an antibody, e.g., of the TfRMAb, and another neurotrophin monomer fused to the second chain (e.g., a light or heavy chain) of the antibody. Typically, the molecular weight range of recombinant proteins that may be fused to the molecular Trojan horse ranges from 1000 Daltons to 500,000 Daltons.

One particularly useful neurotrophin in embodiments of the invention is glial-derived neurotrophic factor (GDNF). GDNF is a powerful neurotherapeutic that can be used to treat motor neuron disease, stroke, alcohol addiction, or drug addiction.

In studies demonstrating the pharmacologic efficacy of GDNF in experimental brain disease, it is necessary to administer the neurotrophin directly into the brain following a transcranial drug delivery procedure. The transcranial drug delivery is required because GDNF does not cross the brain capillary wall, which forms the blood-brain barrier (BBB) in vivo. Owing to the lack of transport of GDNF across the BBB, it is not possible for the neurotrophin to enter the CNS, including the brain or spinal cord, following a peripheral administration unless the BBB is experimentally disrupted. The lack of utility of GDNF as a CNS therapeutic following peripheral administration is expected and follows from the limiting role that is played by the BBB in the development of neurotherapeutics, especially large molecule drugs such as GDNF. GDNF does not cross the BBB, and the lack of transport of the neurotrophin across the BBB prevents the molecule from being pharmacologically active in the brain following peripheral administration. The lack of GDNF transport across the BBB means that the neurotrophin must be directly injected into the brain across the skull bone to be pharmacologically active in the CNS. However, when the GDNF is fused to a Trojan horse such as a mouse TfRMAb, this neurotrophin is now able to enter brain from blood following a non-invasive peripheral route of administration such as intravenous intramuscular, subcutaneous, intraperitoneal, or even oral administration. Owing to the BBB transport properties of this new class of molecule, it is not necessary to administer the GDNF directly into the CNS with an invasive delivery procedure requiring penetration of the skull or spinal canal. The reformulated fusion protein of the GDNF variant and the mouse TfR MAb now enables entry of GDNF into the brain from the blood, and the development of GDNF in mouse models of human diseases.

As used herein, the term “GDNF” includes the pharmaceutically acceptable salts and prodrugs, and prodrugs of the salts, polymorphs, hydrates, solvates, biologically-active fragments, biologically active variants and stereoisomers of the naturally-occurring GDNF, as well as agonist, mimetic, and antagonist variants of the naturally-occurring GDNF and polypeptide fusions thereof. Variants that include one or more deletions, substitutions, or insertions in the natural sequence of the GDNF, in particular truncated versions of the native GDNF comprising deletion of one or more amino acids at the amino terminus, carboxyl terminus, or both, are encompassed by the term “GDNF.” In some embodiments, the invention utilizes a carboxy-truncated variant of the native GDNF, e.g., a variant in which 1, 2, 3, 4, 5, 6, 7, 8, 9, 10, or more than 10 amino acids are absent from the carboxy-terminus of GDNF. GDNF variants include GDNF variants with a truncated amino terminus or carboxy terminus, or variants that comprise an amino acid sequence at least 70%, e.g., at least 75%, 80%, 85%, 87%, 90%, 92%, 95%, or another percent identical from at least 70% to 100% identical to the amino acid sequence of human GDNF, as long as the fusion protein variant still binds to the human GDNF receptor α (GFRα) with high affinity as determined by any standard ligand-receptor binding assay in the art. Examples of such assays include, but are not limited to, ELISA, RIA, cellular reporter assays, or surface plasmon resonance. In some embodiments, fusion protein variants are produced by substitution of amino acids within either the framework region (FR) or the complementarity determining region (CDR) of either the light chain or the heavy chain of the mouse TfRMAb, as long as the fusion protein binds with high affinity to the mouse TfR to promote transport across the mouse BBB. Additional fusion protein variants can be produced by changing the composition or length of a linker polypeptide separating a CNS-active polypeptide (e.g., GDNF) from the mouse TfRMAb. In one embodiment, full-length human GDNF is utilized.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of another peptide. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:17) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

The present invention also includes peptides having a conservative amino acid change, compared with an amino acid sequence disclosed herein. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

It also will be understood that amino acid sequences may include additional residues, such as additional N- or C-terminal amino acids, and yet still be essentially as set forth in one of the sequences disclosed herein, so long as the sequence retains sufficient biological protein activity to be functional in the compositions and methods of the invention

B. Antibodies

One type of CNS-active agents of use in the invention is antibody agents. Many antibody agents, e.g., pharmaceuticals, are active (e.g., pharmacologically active) in brain but do not cross the blood-brain barrier. These factors are suitable for use in the compositions and methods of the invention and include an antibody that is directed against the Aβ amyloid peptide of Alzheimer's disease (AD) for the diagnosis or treatment of AD. In some embodiments, the antibody is directed against α-synuclein of Parkinson's disease (PD) for the diagnosis or treatment of PD. In some embodiments, the antibody is directed against the huntingtin protein of Huntington's disease (HD) for the diagnosis or treatment of HD. In some embodiments, the antibody is directed against the Prp protein of scrapie or mad cow disease for the diagnosis or treatment of human equivalents of scrapie. In some embodiments, the antibody is directed against an envelope protein of the West Nile virus for the diagnosis or treatment of West Nile encephalitis. In some embodiments, the antibody is directed against the tumor necrosis factor (TNF) related apoptosis inducing ligand (TRAIL) for the diagnosis or treatment of acquired immune deficiency syndrome (AIDS), which infects the brain. In some embodiments, the antibody is directed against the nogo A protein for the diagnosis or treatment of brain injury, spinal cord injury, or stroke. In some embodiments, the antibody is directed against the extracellular portion of LINGO-1 for inducing remyelination in regions of CNS that have undergone demyelination due to pathological condition (e.g., multiple sclerosis). In some embodiments, the antibody is directed against the HER2 protein for the diagnosis or treatment of breast cancer metastatic to the brain. In some embodiments, the antibody is directed against oncogenic receptor proteins such as the epidermal growth factor receptor (EGFR) for the diagnosis or treatment of either primary brain cancer or metastatic cancer of the brain. In some embodiments, the antibody is directed against an oncogenic growth factor such as the epidermal growth factor (EGF) or the hepatocyte growth factor (HGF) for the diagnosis or treatment of either primary brain cancer or metastatic cancer of the brain. In some embodiments, the antibody is directed against an oligodendrocyte surface antigen for the diagnosis or treatment of demyelinating disease such as multiple sclerosis. Particularly useful in some embodiments of the invention utilizing ScFv forms of the antibody, e.g., therapeutic antibody, that are used as precursors for fusion proteins that cross the BBB are those that naturally form dimeric structures, similar to original antibody. Some embodiments of the invention provides a fusion protein constructed of ScFv derived from the antibody fused to one chain (e.g., a light or heavy chain) of a mouse TfRMAb.

One particularly useful antibody pharmaceutical in embodiments of the invention is an antibody against the Aβ amyloid peptide of AD. The dementia of AD is caused by the progressive accumulation over many years of amyloid plaque. This plaque is formed by the aggregation of the Aβ amyloid peptide, which is a 40-43 amino acid peptide designated Aβ^{1-40/43}, which is derived from the proteolytic processing within the brain of the amyloid peptide precursor protein called APP.

A potential therapy for AD is any drug that can enter the brain and cause disaggregation of the amyloid plaque. Transgenic mice have been engineered which express mutant forms of the APP protein, and these mice develop amyloid plaque similar to people with AD. The amyloid plaque can be disaggregated with the application of anti-Aβ antibodies administered directly into the brain of the transgenic mice via either direct cerebral injection or via a cranial window. Following anti-Aβ antibody-mediated disaggregation of the amyloid plaque, the dystrophic nerve endings in the vicinity of the plaque begin to heal and form normal structures.

Antibody based therapies of AD include active or passive immunization against the Aβ peptide. In active immunization, the subject is immunized with the Aβ peptide along with an adjuvant such as Freund's adjuvant. Active immunization of transgenic mice resulted in a decrease in the amyloid burden in brain, which is evidence that the anti-Aβ peptide antibodies in the blood formed in the active immunization treatment were able to cross the BBB in the immunized mouse. It is well known that the administration of adjuvants such as Freunds adjuvant causes disruption of the BBB via an inflammatory response to the adjuvant administration. It is likely that active immunization in mouse models of AD will either not be effective, because (a) the adjuvant used is not toxic, and the BBB is not disrupted, or (b) the adjuvant is toxic, and causes opening of the BBB via an inflammatory response to the adjuvant. Opening of the BBB allows the entry into brain of serum proteins such as albumin, and these proteins are toxic to brain cells. In passive immunization, an anti-Aβ peptide antibody is administered directly to the subject with brain amyloid, and this has been done in transgenic mice with brain amyloid similar to AD. However, the dose of anti-Aβ peptide antibody that must be administered to the mice is prohibitively high, owing to the lack of significant transport of antibody molecules in the blood to brain direction. Therefore, the limiting factor in either the active or passive immunization of either transgenic mice or of people with AD and brain amyloid is the BBB, and the lack of transport of antibody molecules across the BBB in the blood to brain direction.

As used herein, the term “anti-Aβ peptide antibody” includes the pharmaceutically acceptable salts, polymorphs, hydrates, solvates, biologically-active fragments, biologically active variants and stereoisomers of the precursor anti-Aβ peptide antibody, as well as agonist, mimetic, and antagonist variants of antibodies directed at alternative targets, which cross-react with the anti-Aβ peptide antibody, and polypeptide fusion variants thereof. Variants include one or more deletions, substitutions, or insertions in the sequence of the anti-Aβ peptide antibody precursor.

In some embodiments, the anti-Aβ peptide antibody is a ScFv antibody comprised of the variable region of the heavy chain (VH) and the variable region of the light chain (VL) derived from a murine anti-Aβ peptide antibody. The amino acid sequence of the HC-ScFv anti-Aβ peptide antibody comprises SEQ ID NO: 21.

Accordingly, anti-Aβ peptide ScFv antibodies useful in the invention include antibodies having at least about 60%, at least about 70%, at least about 80%, at least about 90%, at least about 95%, at least about 99%, or greater than 95% or greater than 99% sequence identity, e.g., 100% sequence identity, to SEQ ID NO:21.

C. Avidin Conjugates

In some embodiments, a CNS-active polypeptide is avidin, avidin, an avidin sequence variant, a chemically modified avidin derivative, streptavidin, a streptavidin sequence variant, or a chemically modified streptavidin derivative complexed with a biotinylated therapeutic agent. Antibody-avidin fusion proteins are described in Penichet et al (1999), J Immunol, 163(8):4421-4426 and in U.S. patent application Ser. No. 10/858,729. The mouse TfRMAb-avidin fusion protein may be complexed with any biotinylated therapeutic agent, for delivery of the biotinylated therapeutic agent across the mouse BBB. In one embodiment, the TfRMAb-avidin fusion protein comprises the rat 8D3 MAb against the mouse TfR. In another embodiment, the TfRMAb-avidin fusion protein comprises a mouse-rat chimeric MAb against the mouse TfR. In another embodiment, the TfRMAb-avidin fusion protein comprises a fully-murinized MAb against the mouse TfR.

Examples of therapeutic agents that may be biotinylated and conjugated with a mouse TfRMAb-avidin fusion protein include, but are not limited to, anti-sense oligonucleotides, RNAi double stranded oligonucleotides, activating RNAa double stranded oligonucleotides (see WO2006113246), plasmid vector DNA, antibodies, neurotrophins, and enzymes.

D. Enzymes

In some embodiments, a CNS-active polypeptide is an enzyme. Examples of suitable enzymes include, but are not limited to, metabolic enzymes, e.g., iduronidase (IDUA). In some embodiments, a CNS-active polypeptide is IDUA. As used herein, IDUA refers to any naturally occurring or artificial enzyme that can catalyze the hydrolysis of unsulfated alpha-L-iduronosidic linkages in dermatan sulfate, e.g., the human IDUA sequence listed under GenBank Accession No. NP_{—}000194.

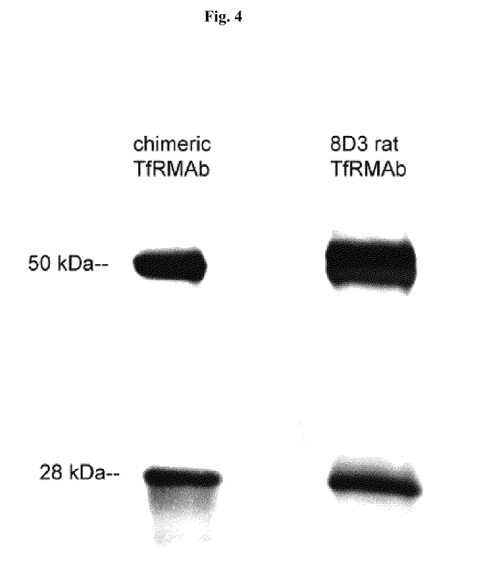

In some embodiments, IDUA has an amino acid sequence that is a at least 50% identical (i.e., at least, 55, 60, 65, 70, 75, 80, 85, 90, 95, or any other percent up to 100% identical) to the amino acid sequence of human IDUA (GenBank No. NP_{—}000194), a 653 amino acid protein listed under GenBank Accession No. NP_{—}000194, or a 627 amino acid subsequence thereof, which lacks a 26 amino acid signal peptide, and corresponds to SEQ ID NO:9 (FIG. 4). The structure-function relationship of human IDUA is well established, as described in, e.g., Rempel et al. (2005), “A homology model for human α-L-Iduronidase: Insights into human disease,” Mol. Genetics and Met., 85:28-37. In particular, residues that are critical to the function of IDUA include, e.g., Gly 51, Ala 75, Ala 160, Glu 182, Gly 208, Leu 218, Asp 315, Ala 327, Asp 349, Thr 366, Thr 388, Arg 489, Arg 628, Ala 79, His 82, Glu 178, Ser 260, Leu 346, Asn 350, Thr 364, Leu 490, Pro 496, Pro 533, Arg 619, Arg 89, Cys 205, His 240, Ala 319, Gln 380, Arg 383, and Arg 492. In some embodiments, the IDUA is fused at its N-terminus to the C-terminus of the cTfRMAb HC or LC. In some embodiments, the IDUA is linked to the C-terminus of the cTfRMAb HC or LC by a short peptide linker of about 2 to 20 amino acids 1, 2, 3, 4, 5, 6, 7, 8, 10, 12, 15, or any other number of amino acids from about to 20 amino acids. In some embodiments, the linker sequence consists of three consecutive serines. A variety of other linkers could be used to join the IgG chain and the therapeutic protein, such as a single amino acid or a dipeptide, or an extended linker could be used. For example, in some embodiments an extended Gly/Ser or GS linker, such as a GGGGSGGGGSGGGGS linker (SEQ ID NO:22), designated GS15, could be introduced at the original short linker to form the extended linker SGGGGSGGGGSGGGGSS (SEQ ID NO:23). Or, a variety of other linkers could be substituted for the short or extended amino acid linkers.

Sequence variants of a canonical IDUA sequence can be generated, e.g., by random mutagenesis of the entire sequence or specific subsequences corresponding to particular domains. Alternatively, site directed mutagenesis can be performed reiteratively while avoiding mutations to residues known to be critical to IDUA function such as those given above. Further, in generating multiple variants of an IDUA sequence, mutation tolerance prediction programs can be used to greatly reduce the number of non-functional sequence variants that would be generated by strictly random mutagenesis. Various programs) for predicting the effects of amino acid substitutions in a protein sequence on protein function (e.g., SIFT, PolyPhen, PANTHER PSEC, PMUT, and TopoSNP) are described in, e.g., Henikoff et al. (2006), “Predicting the Effects of Amino Acid Substitutions on Protein Function,” Annu. Rev. Genomics Hum. Genet., 7:61-80. IDUA sequence variants can be screened for of IDUA activity/retention of IDUA activity by, e.g., 4-methylumbelliferyl α-L-iduronide (MUBI) fluorometric IDUA assays known in the art. See, e.g., Kakkis et al. (1994), Prot Expr Purif 5:225-232. One unit of IDUA activity is defined as the hydrolysis of 1 nmole substrate/hour. Accordingly, one of ordinary skill in the art will appreciate that a very large number of operable IDUA sequence variants can be obtained by generating and screening extremely diverse “libraries” of IDUA sequence variants by methods that are routine in the art, as described above.

### V. Compositions

Compositions of the invention are useful in one or more of: increasing serum half-life of a CNS-active agent (e.g., a CNS-active polypeptide), transporting a CNS-active agent across the BBB, and/or retaining activity of the agent once transported across the BBB. Accordingly, in some embodiments, the invention provides compositions containing a purified monoclonal antibody against the mouse transferrin receptor (e.g., the 8D3 MAb described herein). In some embodiments, a composition comprises a CNS-active agent (e.g., a CNS-active polypeptide) covalently linked to a MAb against the mouse TfR to thereby transport the CNS-active agent across the blood brain barrier (BBB) of a mouse, where the composition is capable of producing an average elevation of concentration in the brain of the neurotherapeutic agent of at least about 1, 2, 3, 4, 5, 10, 20, 30, 40, or 50 ng/gram brain following peripheral administration. The invention also provides compositions containing a CNS-active agent that is covalently linked to a MAb to the mouse TfR, which is transported across the BBB by binding to the TfR on the mouse BBB. In some embodiments, the MAb to the mouse TfR is a Rat-Mouse chimeric MAb against the mouse TfR. The antibody can be glycosylated or non-glycosylated; in some embodiments, the antibody is glycosylated, e.g., in a glycosylation pattern produced by its synthesis in a CHO cell. In some embodiments, the mouse TfRMAb and the CNS-active agent each retain an average of at least about 10, 20, 30, 40, 50, 60, 70, 80, 90, 95, 99, or 100% of their activities, compared to their activities as separate entities. In certain embodiments, the invention further provides compositions that increase the serum half-life of a CNS-active agent, e.g., a CNS-active polypeptide, relative to the serum half-life of the CNS-active agent when administered alone. The invention also provides pharmaceutical compositions that contain one or more compositions of the invention and a pharmaceutically acceptable excipient.

“Elevation” of the CNS-active agent is an increase in the brain concentration of the agent compared to the concentration of the agent administered alone (i.e., not covalently linked to a TfRMAb that crosses the BBB). In the case of agents for which only a small amount of the agent alone normally crosses the BBB, “elevation” may be an increase in the agent compared to basal brain levels. “Average” refers to the mean of at least three, four, five, or more than five measurements, preferably in different individuals. The individual in which the elevation is measured is a mouse or another rodent in which an antibody against the mouse TfR would recognize an endogenous TfR.

The covalent linkage between the antibody and the CNS-active agent may be a linkage between any suitable portion of the antibody and the neurotherapeutic agent, as long as it allows the antibody-agent fusion to cross the blood brain barrier and the CNS-active agent to retain a therapeutically or diagnostically useful portion of its activity within the CNS. In certain embodiments, the covalent link is between one or more light chains of the antibody and the CNS-active agent. In the case of a polypeptide neurotherapeutic agent (e.g., a neurotrophin such as GDNF), the polypeptide can be covalently linked by its carboxy or amino terminus to the carboxy or amino terminus of the light chain (LC) or heavy chain (HC) of the antibody. Any suitable linkage may be used, e.g., carboxy terminus of light chain to amino terminus of CNS-active polypeptide, carboxy terminus of heavy chain to amino terminus of CNS-active polypeptide, amino terminus of light chain to amino terminus of CNS-active polypeptide, amino terminus of heavy chain to amino terminus of CNS-active polypeptide, carboxy terminus of light chain to carboxy terminus of CNS-active polypeptide, carboxy terminus of heavy chain to carboxy terminus of CNS-active polypeptide, amino terminus of light chain to carboxy terminus of CNS-active polypeptide, or amino terminus of heavy chain to carboxy terminus of CNS-active polypeptide. In some embodiments, the linkage is from the carboxy terminus of the HC to the amino terminus of the CNS-active polypeptide. It will be appreciated that a linkage between terminal amino acids is not required, and any linkage which meets the requirements of the invention may be used; such linkages between non-terminal amino acids of peptides are readily accomplished by those of skill in the art.

In some embodiments, the invention utilizes BDNF or a BDNF sequence variant. Strikingly, it has been found that fusion proteins of these forms of BDNF retain full transport and activity. This is surprising because the neurotrophin is translated in vivo in cells as a prepro form and the prepro-BDNF is then converted into mature BDNF following cleavage of the prepro polypeptide from the amino terminus of the BDNF. In order to preserve the prepro form of the BDNF, and the subsequent cleavability of the prepro peptide, it would seem to be necessary to fuse the prepro BDNF to the amino terminus of either the HC or the LC of the targeting MAb. This could be inhibit the binding of the MAb for the target antigen, since the complementarity determining regions (CDR) of the heavy chain or light chain of the MAb molecule, which comprise the antigen binding site of the MAb, are situated near the amino terminus of the heavy chain or light chains of the antibody. Therefore, fusion of the prepro-neurotrophin to the amino terminus of the antibody chains is expected to result in not only impairment of antibody activity, but also an impairment of antibody folding following translation. The present invention shows the unexpected finding that it is possible to fuse the mature form of a neurotrophin, such as a BDNF variant (vBDNF), to the carboxyl terminus of the heavy chain of the TR MAb. The production of this new genetically engineered fusion protein creates a bi-functional molecule that binds with high affinity to both the mouse TR and the trkB receptors.

The covalent linkage between the mouse TfRMAb and the CNS-active agent may be direct (e.g., a polypeptide bond between the terminal amino acid of one polypeptide and the terminal amino acid of the other polypeptide to which it is linked) or indirect, via a linker. If a linker is used, it may be any suitable linker, e.g., a polypeptide linker. If a polypeptide linker is used, it may be 1, 2, 3, 4, 5, 6, 7, 8, 9, 10, or more than 10 amino acids in length. In some embodiments, a three amino acid linker is used. In some embodiments, the linker has the sequence ser-ser-met. The covalent linkage may be cleavable, however this is not a requirement for activity of the system in some embodiments; indeed, an advantage of these embodiments of the present invention is that the fusion protein, without cleavage, is partially or fully active both for transport and for activity once across the BBB.

In some embodiments, a noncovalent attachment may be used. An example of noncovalent attachment of the MTH, e.g., MAb, to the large molecule therapeutic neuroprotective factor is avidin/streptavidin-biotin attachment. Such an approach is further described in U.S. patent application Ser. No. 10/858,729, entitled “Anti-growth factor receptor avidin fusion proteins as universal vectors for drug delivery,” filed Apr. 21, 2005, which is hereby incorporated by reference in its entirety.

The CNS-active agent, e.g., a neurotherapeutic agent, may be any suitable neurotherapeutic agent, such as a neurotrophin. In some embodiments, the neurotherapeutic agent is a neurotrophin such as glial-derived neurotrophic factor (GDNF), brain derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-α, TGF-β, vascular endothelial growth factor (VEGF), interleukin-1 receptor antagonist (IL-1ra), ciliary neurotrophic factor (CNTF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPsi), netrins, saposins, semaphorins, or stem cell factor (SCF). In some embodiments, the neurotrophin is GDNF. The GDNF may be native GDNF or a variant BDNF. The GDNF can be a human GDNF. In some embodiments, the GDNF contains a sequence that is about 60, 70, 80, 85, 90, 95, 99, or 100% identical to the sequence of human GDNF.

In some embodiments, the invention provides compositions containing a fusion MAb, where the fusion MAb is an antibody to the mouse transferrin receptor linked to a CNS-active polypeptide. In some embodiments, the CNS-active polypeptide is linked via its amino terminus to the carboxy terminus of the heavy chain of the antibody by a ser-ser-met linker. In some embodiments the MAb against the mouse TfR is a chimeric antibody with sufficient mouse sequence that it is suitable for administration to a mouse.

Strikingly, it has been found that multifunctional fusion proteins of the invention, e.g., bifunctional fusion proteins, retain a high proportion of the activity of the separate portions, e.g., the portion that is capable of crossing the BBB and the portion that is active in the CNS. Accordingly, the invention further provides a fusion protein containing a mouse TfRMAb that crosses the BBB, covalently linked to a CNS-active polypeptide, where the MAb and the polypeptide that is active in the central nervous system each retain an average of at least about 10%, 20%, 30%, 40%, 50%, 60%, 70%, 80%, 90%, 95%, 99%, or 100% of their activities, compared to their activities as separate entities. In some embodiments, the invention provides a fusion protein containing a mouse TfRMAb covalently linked to a CNS-active polypeptide where the mouse TfR MAb and the CNS-active polypeptide each retain an average of at least about 50% of their activities, compared to their activities as separate entities. In some embodiments, the invention provides a fusion protein containing a mouse TfRMAb covalently linked to a CNS-active polypeptide where the mouse TfR MAb and the CNS-active polypeptide each retain an average of at least about 60% of their activities, compared to their activities as separate entities. In some embodiments, the invention provides a fusion protein containing a mouse TfRMAb covalently linked to a CNS-active polypeptide where the mouse TfR MAb and the CNS-active polypeptide each retain an average of at least about 70% of their activities, compared to their activities as separate entities. In some embodiments, the invention provides a fusion protein containing a mouse TfRMAb covalently linked to a CNS-active polypeptide where the mouse TfR MAb and the CNS-active polypeptide each retain an average of at least about 80% of their activities, compared to their activities as separate entities. In some embodiments, the invention provides a fusion protein containing a mouse TfRMAb covalently linked to a CNS-active polypeptide where the mouse TfR MAb and the CNS-active polypeptide each retain an average of at least about 90% of their activities, compared to their activities as separate entities. In some embodiments, the mouse TfR MAb retains at least about 10, 20, 30, 40, 50, 60, 70, 80, 90, 95, 99, or 100% of its activity, compared to its activity as a separate entity, and the CNS-active polypeptide retains at least about 10, 20, 30, 40, 50, 60, 70, 80, 90, 95, 99, or 100% of its activity, compared to its activity as a separate entity.

As used herein, “activity” includes physiological activity (e.g., ability to cross the BBB and/or therapeutic activity), and also binding affinity for their respective receptors.

Transport of the mouse TfRMAb across the BBB may be compared for the mouse TfRMAb alone and for the mouse TfRMAb as part of a fusion structure of the invention by standard methods. For example, pharmacokinetics and brain uptake of a fusion protein, e.g., fusion in a mouse may be used. Similarly, standard models for the function of an agent, e.g. the therapeutic or protective function of a therapeutic agent, may also be used to compare the function of the CNS-active agent alone and the function of the agent as part of a fusion protein of the invention.

In some embodiments, binding affinity for receptors may be used as a marker of activity. Binding affinity for the receptor is compared for the structure alone and for the structure when part of the fusion protein. A suitable type of binding affinity assay is the competitive ligand binding assay (CLBA). For example, for fusion proteins containing MAbs to endogenous BBB receptor-mediated transport systems fused to a neurotrophin, a CLBA may be used both to assay the affinity of the MAb for its receptor and the neurotrophin for its receptor, either as part of the fusion protein or as separate entities, and percentage affinity calculated. If, as in some embodiments, the polypeptide that is active in the CNS is highly ionic, e.g., cationic, causing a high degree of non-specific binding, suitable measures should be taken to eliminate the nonspecific binding. “Average” measurements are the average of at least three separate measurements.

In certain embodiments, the invention provides compositions that increase the serum half-life of cationic substances. One limitation for many current therapeutics, especially cationic therapeutic polypeptides (e.g., BDNF) is their rapid clearance from the circulation. The positive charge on the cationic substance, such as cationic peptides, rapidly interacts with negative charges on cell membranes, which triggers an absorptive-mediated endocytosis into the cell, particularly liver and spleen. This is true not only for neurotherapeutics (where rapid clearance means only limited contact with the BBB and thus limited ability to cross the BBB) but for other agents as well, such as cationic import peptides such as the tat peptide, or cationic proteins (e.g. protamine, polylysine, polyarginine) that bind nucleic acids, or cationic proteins such as avidin that bind biotinylated drugs. Surprisingly, fusion compositions of the invention that include a cationic therapeutic polypeptide covalently linked to an immunoglobulin show greatly enhanced serum half-life compared to the same polypeptide when it was not covalently part of a fusion immunoglobulin. This is an important finding, because it shows that the fusion of a highly cationic protein, e.g., BDNF, to a mouse TfRMAb, has two important and unexpected effects: 1) it greatly enhances the serum half-life of the cationic protein, and 2) it does not accelerate the blood clearance of the TfRMAb to which it is attached. Prior work shows that the noncovalent attachment of a cationic therapeutic peptide, e.g., the cationic BDNF to a monoclonal antibody greatly accelerated the blood clearance of the antibody, owing to the cationic nature of the BDNF, which greatly enhances hepatic uptake.

Accordingly, in some embodiments, the invention provides composition comprising a cationic therapeutic polypeptide covalently linked to a mouse TfRMAb, wherein the cationic therapeutic polypeptide in the composition has a serum half-life that is an average of at least about 1.5, 2, 3, 4, 5, 6, 7, 8, 9, 10, 15, 20, 30, 40, 50, 60, 70, 80, 90, 100, or more than about 100-fold greater than the serum half-life of the cationic therapeutic polypeptide alone. In some embodiments, the invention provides a composition comprising a cationic therapeutic polypeptide covalently linked to a mouse TfRMAb, wherein the cationic therapeutic polypeptide in the composition has a mean residence time (MRT) in the serum that is an average of at least about 1.5, 2, 3, 4, 5, 6, 7, 8, 9, 10, 15, 20, 30, 40, 50, 60, 70, 80, 90, 100, or more than about 100-fold greater than the serum half-life of the cationic therapeutic polypeptide alone. In some embodiments, the invention provides composition comprising a cationic therapeutic polypeptide covalently linked to a mouse TfRMAb, wherein the cationic therapeutic polypeptide in the composition has a systemic clearance rate that is an average of at least about 1.5, 2, 3, 4, 5, 6, 7, 8, 9, 10, 15, 20, 30, 40, 50, 60, 70, 80, 90, 100, or more than about 100-fold slower than the systemic clearance rate of the cationic therapeutic polypeptide alone. In some embodiments, the invention provides composition comprising a cationic therapeutic polypeptide covalently linked to a mouse TfRMAb, wherein the cationic therapeutic polypeptide in the composition has average blood level after peripheral administration that is an average of at least about 1.5, 2, 3, 4, 5, 6, 7, 8, 9, 10, 15, 20, 30, 40, 50, 60, 70, 80, 90, 100, or more than about 100-fold greater than the average blood level after peripheral administration of the cationic therapeutic polypeptide alone.

In some embodiments, the cationic therapeutic polypeptide comprises a neurotherapeutic agent. Examples of neurotherapeutic agents that are cationic peptides include, but are not limited to, interferons, interleukins, cytokines, or growth factors with an isoelectric point (pI) above 8. In some embodiments, the CNS-active agent is a neurotrophin, an ScFv antibody, or avidin. Cationic polypeptide neurotrophins include BDNF, NT-3, NT-4/5, NGF, and FGF-2. In some embodiments, the neurotrophin is BDNF.

The invention also provides pharmaceutical compositions that contain one or more compositions of the invention and a pharmaceutically acceptable excipient. A thorough discussion of pharmaceutically acceptable carriers/excipients can be found in Remington's Pharmaceutical Sciences, Gennaro, Ariz., ed., 20th edition, 2000: Williams and Wilkins Pa., USA. Pharmaceutical compostions of the invention include compositions suitable for administration via any peripheral route, including intravenous, subcutaneous, intramuscular, intraperitoneal injection; oral, rectal, transbuccal, pulmonary, transdermal, intranasal, or any other suitable route of peripheral administration.

The compositions of the invention are particular suited for injection, e.g., as a pharmaceutical composition for intravenous, subcutaneous, intramuscular, or intraperitoneal administration. Aqueous compositions of the present invention comprise an effective amount of a composition of the present invention, which may be dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to a mouse, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Exemplary pharmaceutically acceptable carriers for injectable compositions can include salts, for example, mineral acid salts such as hydrochlorides, hydrobromides, phosphates, sulfates, and the like; and the salts of organic acids such as acetates, propionates, malonates, benzoates, and the like. For example, compositions of the invention may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate. Under ordinary conditions of storage and use, these preparations can contain a preservative to prevent the growth of microorganisms. The prevention of the action of microorganisms can be brought about by various antibacterial and antifungal agents, for example, parabens, chlorobutanol; phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate, and gelatin.

Preparations meet sterility, pyrogenicity, general safety, and purity standards as required by NIH and animal care guidelines. The active compounds will generally be formulated for parenteral administration, e.g., formulated for injection via the intravenous, intramuscular, subcutaneous, intralesional, or intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed

The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. The person responsible for administration will, in any event, determine the appropriate dose for the individual subject.

The active therapeutic agents may be formulated within a mixture to comprise about 0.0001 to 1.0 milligrams, or about 0.001 to 0.1 milligrams, or about 1.0 to 100 milligrams or even about 0.01 to 1.0 grams per dose or so. Multiple doses can also be administered. In some embodiments, a dosage of about 2.5 to about 25 mg of a fusion protein of the invention is used as a unit dose for administration to a mouse, e.g., about 0.2 to about 10 mg/kg, e.g., about 0.3, 0.4, 0.5, 0.6, 0.7, 1.0, 1.5, 1.6, 2.0, 2.5, 3.0, 4.0, 4.5, 4.7, or another dose from about 0.1 to about 10 mg/kg of a fusion protein of and a mouse TfRMAb and a CNS-active polypeptide, e.g., a neurotrophin.

In addition to the compounds formulated for parenteral administration, such as intravenous or intramuscular injection, other alternative methods of administration of the present invention may also be used, including but not limited to intradermal administration (See U.S. Pat. Nos. 5,997,501; 5,848,991; and 5,527,288), pulmonary administration (See U.S. Pat. Nos. 6,361,760; 6,060,069; and 6,041,775), buccal administration (See U.S. Pat. Nos. 6,375,975; and 6,284,262), transdermal administration (See U.S. Pat. Nos. 6,348,210; and 6,322,808) and transmucosal administration (See U.S. Pat. No. 5,656,284). All such methods of administration are well known in the art. One may also use intranasal administration of the present invention, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and include, for example, antibiotics and antihistamines and are used for asthma prophylaxis.

Additional formulations, which are suitable for other modes of administration, include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids. For suppositories, traditional binders and carriers generally include, for example, polyalkylene glycols or triglycerides; such suppositories may be formed from mixtures containing the active ingredient in any suitable range, e.g., in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations, or powders. In certain defined embodiments, oral pharmaceutical compositions will comprise an inert diluent or assimilable edible carrier, or they may be enclosed in a hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations can contain at least 0.1% of active compound. The percentage of the compositions and preparations may, of course, be varied, and may conveniently be between about 2 to about 75% of the weight of the unit, or between about 25-60%. The amount of active compounds in such therapeutically useful compositions is such that a suitable dosage will be obtained.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent, methylene and propyl parabens as preservatives, a dye and flavoring, such as cherry or orange flavor. In some embodiments, an oral pharmaceutical composition may be enterically coated to protect the active ingredients from the environment of the stomach; enteric coating methods and formulations are well-known in the art.

### VI. Nucleic Acids, Vectors, Cells, and Manufacture

The invention also provides nucleic acids, vectors, cells, and methods of production.

A. Nucleic Acids

In some embodiments, the invention provides nucleic acids that code for polypeptides of the invention. In certain embodiments, the invention provides a single nucleic acid sequence containing a first sequence coding for a light chain of a mouse TfRMAb and second sequence coding a heavy chain of the mouse TfRMAb, where either the first sequence further codes for a CNS-active polypeptide that is expressed as a fusion protein of the CNS-active polypeptide covalently linked to the light chain of the mouse TfRMAb, or the second sequence also codes for a CNS-active polypeptide that is expressed as a fusion protein of the CNS-active polypeptide covalently linked to the heavy chain of the mouse TfRMAb. In some embodiments, the invention provides nucleic acid sequences, and in some embodiments the invention provides nucleic acid sequences that are at least about 60, 70, 80, 90, 95, 99, or 100% identical to a particular nucleotide sequence. For example, in some embodiments, the invention provides a nucleic acid containing a first sequence that is at least about 60, 70, 80, 90, 95, 99, or 100% identical to SEQ ID NO: 13, 16, 20, or its complement. In other embodiments, the inventions provides a nucleic acid comprising a first sequence that encodes an amino acid sequence that is at least 60, 70, 80, 90, 95, 99, or 100% identical to SEQ ID NOs: 14, 15, 17, 19, or 21.

For sequence comparison, of two nucleic acids, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. The BLAST algorithm is typically performed with the “low complexity” filter turned off. The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

In some embodiments, nucleic acids of the invention hybridize specifically under low, medium, or high stringency conditions to the nucleic acid sequence corresponding to SEQ ID NO:13, 16, 20, or its complement. In some embodiments, a nucleic acid of the invention hybridizes specifically under low, medium, or high stringency conditions to a nucleic acid encoding a cTfRMAb HC (SEQ ID NO:14), cTfRMAb LC (SEQ ID NO:15), a cTfRMAb HC-GDNF fusion protein (SEQ ID NO:17), a cTfRMAb HC-avidin fusion protein (SEQ ID NO:19), a cTfRMAb HC-ScFv fusion protein (SEQ ID NO:21), or hybridizes to the complement of such a nucleic acid. Low stringency hybridization conditions include, e.g., hybridization with a 100 nucleotide probe of about 40% to about 70% GC content; at 42° C. in 2×SSC and 0.1% SDS. Medium stringency hybridization conditions include, e.g., at 50° C. in 0.5×SSC and 0.1% SDS. High stringency hybridization conditions include, e.g., hybridization with the above-mentioned probe at 65° C. in 0.2×SSC and 0.1% SDS. Under these conditions, as the hybridization temperature is elevated, a nucleic acid with a higher homology can be obtained.

The invention provides nucleic acids that code for any of the polypeptides of the invention. In some embodiments, the invention provides a single nucleic acid sequence containing a gene coding for a light chain of a mouse TfRMAb and a gene coding for a fusion protein, where the fusion protein includes a heavy chain of the mouse TfRMAb covalently linked to a CNS-active polypeptide. In some embodiments, the polypeptide is a therapeutic peptide. In some embodiments the polypeptide is a neurotherapeutic polypeptide, e.g., a neurotrophin such as BDNF. In some embodiments, the BDNF is a two amino acid carboxy-truncated BDNF. Any suitable polypeptide, neurotherapeutic polypeptide, neurotrophin, GDNF, BDNF, avidin, ScFv, antibody, monoclonal antibody, or chimeric antibody, as described herein, may be coded for by the nucleic acid, combined as a fusion protein and coded for in a single nucleic acid sequence. As is well-known in the art, owing to the degeneracy of the genetic code, any combination of suitable codons may be used to code for the desired fusion protein. In addition, other elements useful in recombinant technology, such as promoters, termination signals, and the like, may also be included in the nucleic acid sequence. Such elements are well-known in the art. In addition, all nucleic acid sequences described and claimed herein include the complement of the sequence.

In some embodiments where the nucleic acid codes for a GDNF, e.g., a sequence variant of human GDNF, as a component of the fusion protein. In some embodiments, the nucleic acid encodes an amino acid sequence at least 60, 70, 80, 85, 90, 95, 99, or 100% identical to SEQ ID NO:17. In some embodiments, the GDNF is linked at its amino terminus to carboxy terminus of the heavy chain of the immunoglobulin, e.g., MAb. In on embodiment, the heavy chain of the TfR MAb comprises a sequence that is about 60, 70, 80, 90, 95, 99 or 100% identical to SEQ ID NO:14. In some embodiments, the light chain of the TfRMAb, comprises a sequence that is about 60, 70, 80, 90, 95, 99 or 100% identical to SEQ ID NO:15. The nucleic acid can further contain a nucleic acid sequence that codes for a polypeptide linker between the heavy chain of the MAb and the GDNF. In some embodiments, the linker is S—S-M. The nucleic acid may further contain a nucleic acid sequence coding for a signal peptide, wherein the signal peptide is linked to the heavy chain. Any suitable signal peptide, as known in the art or subsequently developed, may be used.

In certain embodiments, the invention provides a nucleic acid comprising a first sequence that codes for a neurotherapeutic polypeptide, e.g., a neurotrophin such as BDNF, in the same open reading frame as a second sequence that codes for an immunoglobulin component. The immunoglobulin component can be, e.g., a light chain or a heavy chain, e.g., that is at least about 60, 70, 80, 90, 95, 99, or 100% identical SEQ ID NOs 14 or 15. In some embodiments, the nucleic acid further contains a sequence that codes for a selectable marker, such as dihydrofolate reductase (DHFR).

B. Vectors

The invention also provides vectors. The vector can contain any of the nucleic acid sequences described herein. In some embodiments, the invention provides a single tandem expression vector containing nucleic acid coding for a mouse TfRMAb heavy chain fused to a CNS-active polypeptide, e.g., a therapeutic polypeptide such as a neurotrophin, and nucleic acid coding for a light chain of the antibody, all incorporated into a single piece of nucleic acid, e.g., a single piece of DNA. The single tandem vector can also include one or more selection and/or amplification genes. A method of making an exemplary vector of the invention is provided in the Examples. However, any suitable techniques, as known in the art, may be used to construct the vector.

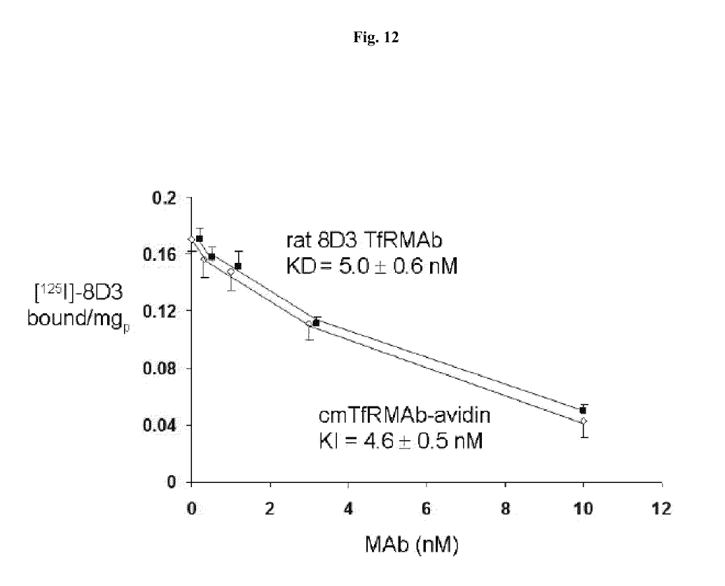

The use of a single tandem vector has several advantages over previous techniques. The transfection of a eukaryotic cell line with immunoglobulin G (IgG) genes generally involves the co-transfection of the cell line with separate plasmids encoding the heavy chain (HC) and the light chain (LC) comprising the IgG. In the case of a IgG fusion protein, the gene encoding the recombinant therapeutic protein may be fused to either the HC or LC gene. However, this co-transfection approach makes it difficult to select a cell line that has equally high integration of both the HC and LC-fusion genes, or the HC-fusion and LC genes. The approach to manufacturing the fusion protein utilized in certain embodiments of the invention is the production of a cell line that is permanently transfected with a single plasmid DNA that contains all the required genes on a single strand of DNA, including the HC-fusion protein gene, the LC gene, the selection gene, e.g. neo, and the amplification gene, e.g. the dihydrofolate reductase gene. As shown in the diagram of the fusion protein tandem vector in FIG. 12, the HC-fusion gene, the LC gene, the neo gene, and the DHFR gene are all under the control of separate, but tandem promoters and separate but tandem transcription termination sequences. Therefore, all genes are equally integrated into the host cell genome, including the fusion gene of the therapeutic protein and either the HC or LC IgG gene.

C. Cells

The invention further provides cells that incorporate one or more of the vectors of the invention. The cell may be a prokaryotic cell or a eukaryotic cell. In some embodiments, the cell is a eukaryotic cell. In some embodiments, the cell is a mouse myeloma hybridoma cell. In some embodiments, the cell is a Chinese hamster ovary (CHO) cell. Exemplary methods for incorporation of the vector(s) into the cell are given in the Examples. However, any suitable techniques, as known in the art, may be used to incorporate the vector(s) into the cell. In some embodiments, the invention provides a cell capable of expressing an immunoglobulin fusion protein, where the cell is a cell into which has been permanently introduced a single tandem expression vector, where both the immunoglobulin light chain gene and the gene for the immunoglobulin heavy chain fused to the therapeutic agent, are incorporated into a single piece of nucleic acid, e.g., DNA. In some embodiments, the invention provides a cell capable of expressing an immunoglobulin fusion protein, where the cell is a cell into which has been permanently introduced a single tandem expression vector, where both the immunoglobulin heavy chain gene and the gene for the immunoglobulin light chain fused to the therapeutic agent, are incorporated into a single piece of nucleic acid, e.g., DNA. The introduction of the tandem vector may be by, e.g., permanent integration into the chromosomal nucleic acid, or by, e.g., introduction of an episomal genetic element.

D. Manufacture

In addition, the invention provides methods of manufacture. In some embodiments, the invention provides a method of manufacturing an immunoglobulin fusion protein, where the fusion protein contains an immunoglobulin heavy chain fused to a therapeutic agent, by permanently introducing into a eukaryotic cell a single tandem expression vector, where both the immunoglobulin light chain gene and the gene for the immunoglobulin heavy chain fused to the CNS-active polypeptide, are incorporated into a single piece of nucleic acid, e.g., DNA. In some embodiments, the invention provides a method of manufacturing a mouse TfRMAb fusion protein, where the fusion protein contains an immunoglobulin light chain fused to a therapeutic agent, by permanently introducing into a eukaryotic cell a single tandem expression vector, where both the immunoglobulin heavy chain gene and the gene for the immunoglobulin light chain fused to the therapeutic agent, are incorporated into a single piece of nucleic acid, e.g., DNA. In some embodiments, the introduction of the vector is accomplished by permanent integration into the host cell genome. In some embodiments, the introduction of the vector is accomplished by introduction of an episomal genetic element containing the vector into the host cell. Episomal genetic elements are well-known in the art. In some embodiments, the therapeutic agent is a neurotherapeutic agent. In some embodiments, the single piece of nucleic acid further includes one or more genes for selectable markers. In some embodiments, the single piece of nucleic acid further includes one or more amplification genes. In some embodiments, the mouse TfRMAb MAb is a chimeric MAb. The methods may further include expressing the immunoglobulin fusion protein, and/or purifying the immunoglobulin fusion protein. Exemplary methods for manufacture, including expression and purification, are given in the Examples.

Suitable techniques, as known in the art, may be used to manufacture, optionally express, and purify the proteins. These include non-recombinant techniques of protein synthesis, such as solid phase synthesis, manual or automated, as first developed by Merrifield and described by Stewart et al. in Solid Phase polypeptide Synthesis (1984). Chemical synthesis joins the amino acids in the predetermined sequence starting at the C-terminus. Basic solid phase methods require coupling the C-terminal protected α-amino acid to a suitable insoluble resin support. Amino acids for synthesis require protection on the α-amino group to ensure proper polypeptide bond formation with the preceding residue (or resin support). Following completion of the condensation reaction at the carboxyl end, the α-amino protecting group is removed to allow the addition of the next residue. Several classes of α-protecting groups have been described, see Stewart et al. in Solid Phase polypeptide Synthesis (1984), with the acid labile, urethane-based tertiary-butyloxycarbonyl (Boc) being the historically preferred. Other protecting groups, and the related chemical strategies, may be used, including the base labile 9-fluorenylmethyloxycarbonyl (FMOC). Also, the reactive amino acid sidechain functional groups require blocking until the synthesis is completed. The complex array of functional blocking groups, along with strategies and limitations to their use, have been reviewed by Bodansky in polypeptide Synthesis (1976) and, Stewart et al. in Solid Phase polypeptide Synthesis (1984).

Solid phase synthesis is initiated by the coupling of the described C-terminal α-protected amino acid residue. Coupling requires activating agents, such as dicyclohexycarbodiimide (DCC) with or without 1-hydroxybenzo-triazole (HOBT), diisopropylcarbodiimide (DIIPC), or ethyldimethylaminopropylcarbodiimide (EDC). After coupling the C-terminal residue, the α-amino protected group is removed by trifluoroacetic acid (25% or greater) in dichloromethane in the case of acid labile tertiary-butyloxycarbonyl (Boc) groups. A neutralizing step with triethylamine (10%) in dichloro-methane recovers the free amine (versus the salt). After the C-terminal residue is added to the resin, the cycle of deprotection, neutralization and coupling, with intermediate wash steps, is repeated in order to extend the protected polypeptide chain. Each protected amino acid is introduced in excess (three to five fold) with equimolar amounts of coupling reagent in suitable solvent. Finally, after the completely blocked polypeptide is assembled on the resin support, reagents are applied to cleave the polypeptide form the resin and to remove the side chain blocking groups. Anhydrous hydrogen fluoride (HF) cleaves the acid labile tertiary-butyloxycarbonyl (Boc) chemistry groups. Several nucleophilic scavengers, such as dimethylsulfide and anisole, are included to avoid side reactions especially on side chain functional groups.

### VII. Recombinant Mice

The present invention also provides a recombinant mouse (e.g., a transgenic mouse disease model), where a mouse has been administered a fusion protein described herein, e.g., the HC or LC of a mouse chimeric TfRMAb (a chimeric antibody to the mouse TfR) fused at the C-terminus of a HC to the N-terminus of a neurotrophin. In some embodiments, the mouse is a model of a human pathological condition, e.g., a CNS condition. In one embodiment, the mouse chimeric TfRMAb is a rat-mouse chimeric TfRMAb. In some embodiments, the chimeric TfRMAb is at least 70, 75, 80, 85, 90, 95, 97, 98, or 99% mouse sequence. In one embodiment, the chimeric TfRMAb is a fully murine MAb. In some embodiments, a recombinant mouse comprises one or more exogenous nucleic acids encoding a mouse TfRMAb HC- or LC fused to a CNS-active polypeptide and a mouse cTfRMAb-LC or HC so that cTfRMAb fusion antibodies are secreted from the cells of the recombinant mouse. In some embodiments, a recombinant mouse comprises one or more exogenous nucleic acids encoding any of the polypeptides of the present invention, so that antibodies are secreted from cells of the recombinant mouse. In some embodiments, a recombinant mouse comprises one or more exogenous nucleic acids encoding a mouse cTfRMAb HC fused to a CNS-active polypeptide and a mouse cTfRMAb-LC so that cTfRMAb fusion antibodies are secreted from cells of the recombinant mouse. In some embodiments, a recombinant mouse comprises one or more exogenous nucleic acids encoding a mouse cTfRMAb LC fused to a CNS-active polypeptide and a mouse cTfRMAb-HC so that cTfRMAb fusion antibodies are secreted from cells of the recombinant mouse. In some embodiments the exogenous nucleic acids are integrated into the genome of the recombinant mouse. In some embodiments, the exogenous nucleic acids are part of an expression vector (e.g., a viral vector) introduced into the mouse.

A number of mouse disease models are useful in the present invention. Examples of suitable mouse disease models include, but are not limited to, transgenic mouse models of progressive neurodegenerative diseases (PNDs), e.g., Alzheimer's disease, and amylotrophic lateral sclerosis) have been established. See, e.g., Spires et al. (2005), NeuroRx., 2(3):447-64 and Wong et al. (2002), Nat. Neurosci., 5(7):633-639. Such transgenic animal models spontaneously develop a PND that is manifested behaviorally by impaired learning, memory, or locomotion. Such animal models are suitable for administration of the compositions described herein.

A PND can also be induced in a non-human mammal by non-genetic means. For example, a PND that affects learning and memory can be induced in a rodent by injecting aggregated Aβ peptide intracereberally as described in, e.g., Yan et al. (2001), Br. J. Pharmacol., 133(1):89-96.

Cognitive abilities, as well as motor functions in non-human animals suffering from a PND, can be assessed using a number of behavioral tasks. Well-established sensitive learning and memory assays include the Morris Water Maze (MWM), context-dependent fear conditioning, cued-fear conditioning, and context-dependent discrimination. See, e.g., Anger (1991), Neurotoxicology, 12(3):403-413. Examples of motor behavior/function assays, include the rotorod test, treadmill running, and general assessment of locomotion . . .

### VIII. Methods

The invention also provides methods. In some embodiments, the invention provides methods for delivery of a CNS-active agent across the mouse BBB in an effective amount. In some embodiments, the invention provides therapeutic, diagnostic, or research methods. Diagnostic methods include the development of polypeptide radiopharmaceuticals capable of transport across the BBB, such as the fusion of a polypeptide ligand, or peptidomimetic MAb for an endogenous receptor in the brain, followed by the radiolabelling of the fusion protein, followed by systemic administration, and external imaging of the localization within the brain of the polypeptide radiopharmaceutical.

Neurotrophin drug development illustrates the problems encountered when development of the delivery of agents active in the CNS, e.g., CNS drug development, is undertaken in the absence of a parallel program in delivery across the BBB, e.g., CNS drug delivery. The advances in the molecular neurosciences during the Decade of the Brain of the 1990s led to the cloning, expression and purification of more than 30 different neurotrophic factors, including BDNF, nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-α, TGF-β, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, or stem cell factor (SCF). These natural substances are powerful restorative agents in the brain and produce neuroprotection when the protein is injected directly into the brain. In addition, the direct injection of BDNF into the brain is a potent stimulant to new brain cell formation and neurogenesis.

Neurotrophins such as BDNF must be injected directly into the brain to achieve a therapeutic effect, because the neurotrophin does not cross the BBB. Therefore, it is not expected that neurotrophic factors will have beneficial effects on brain disorders following the peripheral (intravenous, subcutaneous) administration of these molecules. During the 1990s, there were attempts to develop neurotrophic factors for the treatment of a chronic neurodegenerative disorder, amyotrophic lateral sclerosis (ALS). The clinical protocols administered the neurotrophic factor by subcutaneous administration, even though the neurotrophin must pass the BBB to be therapeutic in neurodegenerative disease. The clinical trials went forward and all neurotrophin phase III clinical trials for ALS failed. Subsequently, attempts were made to administer neurotrophins via intra-cerebroventricular (ICV) infusion, or convection enhanced diffusion (CED), but these highly invasive modes of delivery were either ineffective or toxic. Given the failure of neurotrophin molecules, per se, as neurotherapeutics, more recent theories propose the development of neurotrophin small molecule mimetics, neurotrophin gene therapy, or neurotrophin stem cell therapy.

However, neurotherapeutics can be developed as drugs for peripheral routes of administration, providing the neurotherapeutic is enabled to cross the BBB. Attachment of the neurotherapeutic, e.g. a neurotrophin such as BDNF to a MTH, e.g., the chimeric TfRMAb provides non-invasive delivery of neurotherapeutics to the CNS in animals, e.g., experimental mouse models of acute brain and spinal cord conditions, such as focal brain ischemia, global brain ischemia, and spinal cord injury, and chronic treatment of neurodegenerative disease, including prion diseases, Alzheimer's disease (AD), Parkinson's disease (PD), Huntington's disease (HD), ALS, multiple sclerosis, transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, and retinal degeneration.

Accordingly, in some embodiments the invention provides methods of transport of a CNS-active agent from the peripheral circulation across the BBB in an effective amount, where the CNS-active agent is covalently attached to a mouse TfRMAb that crosses the BBB, and where the CNS-active agent alone is not transported across the BBB in an effective amount.

The invention also provides, in some embodiments, methods of treatment of disorders of the CNS by peripheral administration of an effective amount of a therapeutic agent, e.g., a neurotherapeutic agent covalently linked to a moue TfRMAb that crosses the BBB, where the agent alone is not capable of crossing the BBB in an effective amount when administered peripherally. In some embodiments, the CNS disorder is an acute disorder, and, in some cases, may require only a single administration of the agent. In some embodiments, the CNS disorder is a chronic disorder and may require more than one administration of the agent.

In some embodiments, the effective amount, e.g., therapeutically effective amount is such that a concentration in the brain is reached of at least about 0.001, 0.01, 0.1, 0.5, 1, 2, 3, 4, 5, 6, 7, 8, 9, 10, 15, 20, 25, 30, 35, 40, 45, 50, 100, or more than 100 ng/gram brain. In some embodiments, a therapeutically effective amount, e.g., of a neurotrophin such as GDNF, is such that a brain level is achieved of about 0.1 to 1000, or about 1-100, or about 5-50 ng/g brain. In some embodiments, the neurotherapeutic agent is a neurotrophin. In some embodiments, the neurotrophin is selected from the group consisting of BDNF, nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-α, TGF-β, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, or stem cell factor (SCF). In some embodiments, the neurotrophin is GDNF.

In some embodiments, the invention provides methods of treating a disorder of the CNS in a mouse (e.g., a disease model mouse) by peripherally administering to an individual in need of such treatment an effective amount of a neurotrophin, where the neurotrophin is capable of crossing the BBB to produce an average elevation of neurotrophin concentration in the brain of at least about 0.1, 1, 2, 3, 4, 5, 6, 7, 8, 9, 10, 15, 20, 25, 30, 35, 40, 45, 50, 100, or more than 100 ng/gram brain following said peripheral administration, and where the neurotrophin remains at the elevated level for about 1, 2, 3, 4, 5, 6, 7, 8, 9, 10, or more than 10 days after a single administration. In some embodiments, the neurotrophin remains at a level of greater than about 1 ng/g brain, or about 2 ng/g brain, or about 5 ng/g brain for about 2, 3, 4, 5, 6, 7, 8, 9, 10, or more than 10 days after a single administration.

In some embodiments, the invention provides methods of treating a disorder of the CNS by peripherally administering to an animal in need of such treatment an effective amount of a composition of the invention. The term “peripheral administration,” as used herein, includes any method of administration that is not direct administration into the CNS, i.e., that does not involve physical penetration or disruption of the BBB. “Peripheral administration” includes, but is not limited to, intravenous intramuscular, subcutaneous, intraperitoneal, intranasal, transbuccal, transdermal, rectal, transalveolar (inhalation), or oral administration. Any suitable composition of the invention, as described herein, may be used. In some embodiments, the composition is a neurotrophin covalently linked to a chimeric mouse TfR-MAb. In some embodiments, the neurotrophin is GDNF.

A “disorder of the CNS” or “CNS disorder,” as those terms are used herein, encompasses any condition that affects the brain and/or spinal cord and that leads to suboptimal function. In some embodiments, the disorder is an acute disorder. Acute disorders of the CNS include focal brain ischemia, global brain ischemia, brain trauma, spinal cord injury, acute infections, status epilepticus, migraine headache, acute psychosis, suicidal depression, and acute anxiety/phobia. In some embodiments, the disorder is a chronic disorder. Chronic disorders of the CNS include chronic neurodegeneration, retinal degeneration, depression, chronic affective disorders, lysosomal storage disorders, chronic infections of the brain, brain cancer, stroke rehabilitation, inborn errors of metabolism, autism, mental retardation. Chronic neurodegeneration includes neurodegenerative diseases such as prion diseases, Alzheimer's disease (AD), Parkinson's disease (PD), Huntington's disease (HD), multiple sclerosis (MS), amyotrophic lateral sclerosis (ALS), transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, and retinal degeneration, and aging of the CNS.

In some embodiments, the invention provides methods of treatment of the retina, or for treatment or prevention of blindness. The retina, like the brain, is protected from the blood by the blood-retinal barrier (BRB). The transferrin receptor is expressed on both the BBB and the BRB, and the TfRMAb has been shown to deliver therapeutics to the retina via RMT across the BRB. BDNF is neuroprotective in retinal degeneration, but it was necessary to inject the neurotrophin directly into the eyeball, because BDNF does not cross the BRB. In some embodiments, fusion proteins of the invention are used to treat retinal degeneration and blindness with a route of administration no more invasive than an intravenous or subcutaneous injection, because the TfRMAb delivers the BDNF across the BRB, so that the neurotrophin is exposed to retinal neural cells from the blood compartment.

Any suitable formulation, route of administration, and dose of the compositions of the invention may be used. Formulations, doses, and routes of administration are determined by those of ordinary skill in the art with no more than routine experimentation. Compositions of the invention, e.g., fusion proteins are typically administered in a single dose, e.g., an intravenous dose, of about 0.01-1000 mg, or about 0.05-500 mg, or about 0.1-100 mg, or about 1-100 mg, or about 0.5-50 mg, or about 5-50 mg, or about 1, 2, 3, 4, 5, 6, 7, 8, 9, 10, 11, 12, 15, 20, 25, 30, 25, 40, 45, 50, 60, 70, 80, 90, or 100 mg. Typically, for the treatment of acute brain disease, such as stroke, cardiac arrest, spinal cord injury, or brain trauma, higher doses may be used, whereas for the treatment of chronic conditions such as Alzheimer's disease, Parkinson's disease, Huntington's disease, MS, ALS, transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, and retinal degeneration, and aging, lower, chronic dosing may be used. Oral administration can require a higher dosage than intravenous or subcutaneous dosing, depending on the efficiency of absorption and possible metabolism of the protein, as is known in the art, and may be adjusted from the foregoing based on routine experimentation.

For intravenous or subcutaneous administration, formulations of the invention may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate.

Dosages of the compositions described herein may range from about 2 to about 200 μg/kg in the mouse.

The fusion proteins described herein may also be formulated for chronic use for the treatment of a chronic CNS disorder, e.g., neurodegenerative disease, stroke or brain/spinal cord injury rehabilitation, or depression. Chronic treatment may involve daily, weekly, bi-weekly administration of the composition of the invention, e.g., fusion protein either intravenously, intra-muscularly, or subcutaneous in formulations similar to that used for acute treatment. Alternatively, the composition, e.g., fusion protein may be formulated as part of a bio-degradable polymer, and administered on a monthly schedule.

The composition of the invention, e.g., fusion protein may be administered as part of a combination therapy. The combination therapy involves the administration of a composition of the invention in combination with another therapy for the CNS disorder being treated. If the composition of the invention is used in combination with another CNS disorder method or composition, any combination of the composition of the invention and the additional method or composition may be used. Thus, for example, if use of a composition of the invention is in combination with another CNS disorder treatment agent, the two may be administered simultaneously, consecutively, in overlapping durations, in similar, the same, or different frequencies, etc. In some cases a composition will be used that contains a composition of the invention in combination with one or more other CNS disorder treatment agents.

Other CNS disorder treatment agents that may be used in methods of the invention include, without limitation, thrombolytic therapy for stroke, amyloid-directed therapy for Alzheimer's disease, dopamine restoration therapy for Parkinsons disease, RNA interference therapy for genetic disorders, cancer, or infections, and anti-convulsant therapy for epilepsy. Dosages, routes of administration, administration regimes, and the like for these agents are well-known in the art.

In some embodiments, the composition, e.g., fusion protein is co-administered to the patient with another medication, either within the same formulation or as a separate composition. For example, the fusion protein could be formulated with another fusion protein that is also designed to deliver across the mouse blood-brain barrier a recombinant protein other than GDNF. The fusion protein may be formulated in combination with other large or small molecules.

### IX. EXAMPLES

### Example 1

### Genetic Engineering and Expression of a Mouse/Rat Chimeric Monoclonal Antibody Against the Mouse Transferrin Receptor

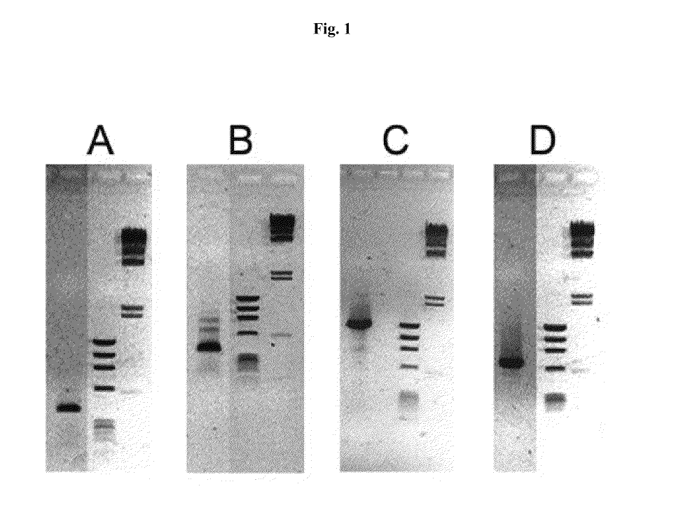

PCR cloning of 8D3 VH and VL, mouse IgG1 heavy chain C-region region, and mouse kappa light chain C-region region. cDNA was produced by reverse transcription of RNA isolated from cultured hybridoma cells. RNA was isolated from 2 different hybridomas: the rat hybridoma producing the 8D3 MAb and a mouse hybridoma producing a MAb comprised of a mouse IgG1 (mIgG1) heavy chain (HC) and a mouse kappa (mKappa) light chain (LC). The cDNAs corresponding to the 4 genes were cloned by the polymerase chain reaction (PCR) using the forward and reverse oligodexoynucleotide (ODN) primers (0.2 uM) described in Table 1, 25 ng polyA+RNA-derived cDNA, 0.2 mM deoxynucleosidetriphosphates, and 2.5 U PfuUltra DNA polymerase in a 50 μl Pfu buffer. SEQ ID NO. 1 and 2, 3 and 4, 5 and 6, 7 and 8, were used to clone the VH, the VL, the HC constant region, and the LC constant region, respectively (Table 1). The amplification was performed in a Mastercycler temperature cycler with an initial denaturing step of 95° C. for 2 min followed by 30 cycles of denaturing at 95° C. for 30 sec, annealing at 55° C. for 30 sec and amplification at 72° C. for 2 min; followed by a final incubation at 72° C. for 10 min. PCR products were resolved with 0.8% agarose gel electrophoresis. A single PCR product was isolated for all 4 genes. An 0.4 kb cDNA was isolated for the 8D3 VH (FIG. 1A); an 0.4 kb cDNA was isolated for 8D3 VL (FIG. 1B); a 1.4 kb cDNA was isolated for the mouse IgG1 HC C-region (FIG. 1C); and an 0.7 kb cDNA was isolated for the mouse K LC C-region (FIG. 1D). These 4 cDNAs were subcloned into the pCR-Script plasmid and subjected to DNA sequence analysis, which allowed for deduced amino acid sequences.

Amino acid micro-sequencing of 8D3 heavy and light chains. The amino terminal amino acid sequences of the 8D3 heavy chain and light chain were determined to (a) confirm isolation of the correct cDNAs encoding the VH and VL, and (b) identify any errors in the amino terminal sequences caused by degeneracy in the PCR primers. The 8D3 hybridoma was cultured, and the 8D3 MAb was purified by protein G affinity chromatography. The 8D3 MAb was applied to a 12% sodium dodecyl sulfate polyacrylamide gel electrophoresis (SDS-PAGE) followed by blotting to a PVDF filter and amino acid microsequencing analysis at the amino terminus was performed. The HC was sequenced through the first 11 amino acids and the LC was sequenced through the first 21 amino acids. This N-terminal amino acid microsequence data matched with the predicted amino acid sequence derived from the cloning of the 8D3 VH and 8D3 VL genes, and revealed PCR-induced errors in the amino terminal amino acid sequence in both the VH and VL. In engineering of the expression plasmids described below, custom PCR primers were design to introduce a V3Q and L4M change in the VL and a Q5V change in the VH sequence, using standard site-directed mutagenesis techniques. Apart from these PCR-induced changes, there was a 100% match in amino acid identity between the predicted and observed amino acid sequence of the 8D3 HC and LC amino termini.

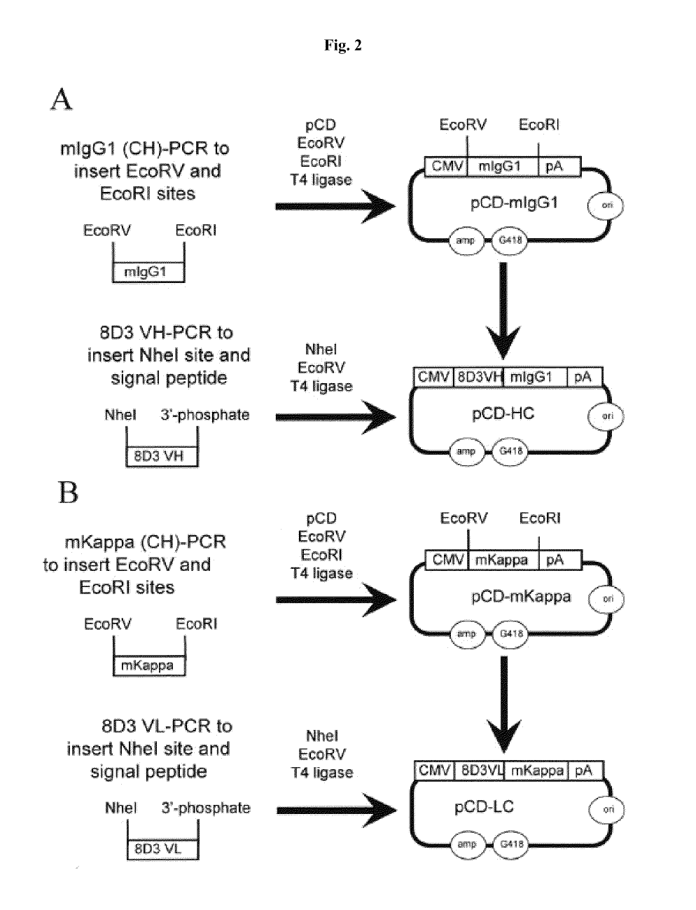

Engineering of chimeric TfRMAb expression plasmid DNAs. The engineering of the chimeric TfRMAb (cTfRMAb) HC expression plasmid, pCD-HC, was completed as outlined in FIG. 2A. The mIgG1 C-region cDNA was generated by PCR using the pCR-Script described above as template, and to introduce both EcoRV and EcoRI sites at the 5′- and 3′-ends, respectively. The mIgG1 C-region cDNA was inserted at the same restriction endonuclease site of the expression plasmid pcDNA3.1 to form the intermediate plasmid pCD-mIgG1 (FIG. 2A). The engineering of the cTfRMAb HC expression plasmid was then completed by insertion of the 8D3 VH cDNA into the NheI and EcoRV sites of pCD-mIgG1 to form pCD-HC (FIG. 2A). The 8D3 VH cDNA was generated by PCR and it has both a NheI site and the signal peptide sequence on the 5′-end. The 3′-end of the PCR generated 8D3 VH was phosphorylated for ligation into the EcoRV site of pCD-mIgG1.

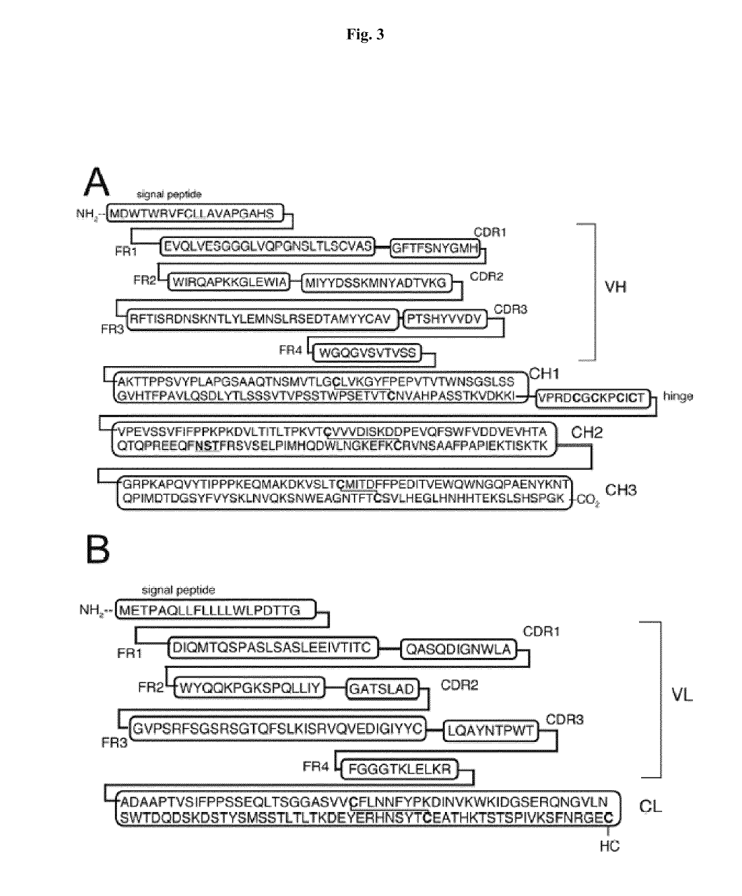

The engineering of the cTfRMAb LC expression plasmid, pCD-LC, was completed as outlined in FIG. 2B. The C-region of the mouse kappa (mKappa) cDNA was generated by PCR using the pCR-Script described above as template, and to introduce both EcoRV and EcoRI sites at the 5′- and 3′-ends, respectively. The mKappa constant C-region cDNA was inserted at the same restriction endonuclease site of the expression plasmid pcDNA3.1 to form the intermediate plasmid pCD-mKappa (FIG. 2B). The engineering of the cTfRMAb LC expression plasmid was then completed by insertion of the 8D3 VL cDNA into the NheI and EcoRV sites of pCD-mKappa to form pCD-LC (FIG. 2B). The 8D3 VL cDNA was generated by PCR and it has both a NheI site and a signal peptide sequence on the 5′-end. The 3′-end of the PCR generated 8D3 VL was phosphorylated for ligation into the EcoRV site of pCD-mKappa. All intermediate plasmids were treated with alkaline phosphatase to prevent self-ligation. Constructs were subjected to nucleotide sequence analysis in both directions. The DNA sequence of the HC and LC genes allowed for the determination of the deduced amino acid (AA) sequence of the HC and LC of the cTfRMAb. The AA sequence, and domain structure, of the HC of the cTfRMAb is shown in FIG. 3A, and the AA sequence, and domain structure, of the LC of the cTfRMAb is shown in FIG. 3B.

Transient expression of chimeric TfRMAb in COS cells. COS cells were dual transfected with pCD-LC and pCD-HC using Lipofectamine 2000, with a ratio of 1:2.5 μg DNA:uL Lipofectamine. Following transfection, the cells were cultured in serum free medium. The conditioned serum free medium was collected at 3 and 7 days. The cTfRMAb, which contained the mouse IgG1 C-region, was purified by affinity chromatography with a 5 mL column of protein G Sepharose 4 Fast Flow.

Mouse IgG-specific ELISA and Western blot. The secretion of the cTfRMAb into the transfected COS cell conditioned serum free medium was detected with a mouse IgG-specific ELISA. The primary antibody was a goat anti-mouse IgG1 Fc region antibody, which was plated at 0.4 μg/well in 96 well plates. The binding of the cTfRMAb to the primary antibody was detected with a conjugate of alkaline phosphatase and a goat anti-mouse LC kappa antibody. Mouse IgG1/kappa IgG was used as the assay standard. The cTfRMAb was detected by Western blotting. Both the chimeric TfRMAb expressed in COS cells, and the hybridoma generated 8D3 TfRMAb, were spotted to 12% SDS-PAGE gels under reducing conditions, following by blotting to nitrocellulose. The primary antibody was a goat anti-mouse IgG (H+L) antibody and the secondary antibody was a biotinylated horse anti-goat IgG. As shown in FIG. 4, the chimeric TfRMAb and the 8D3 hybridoma generated TfRMAb reacted equally in the Western blotting.

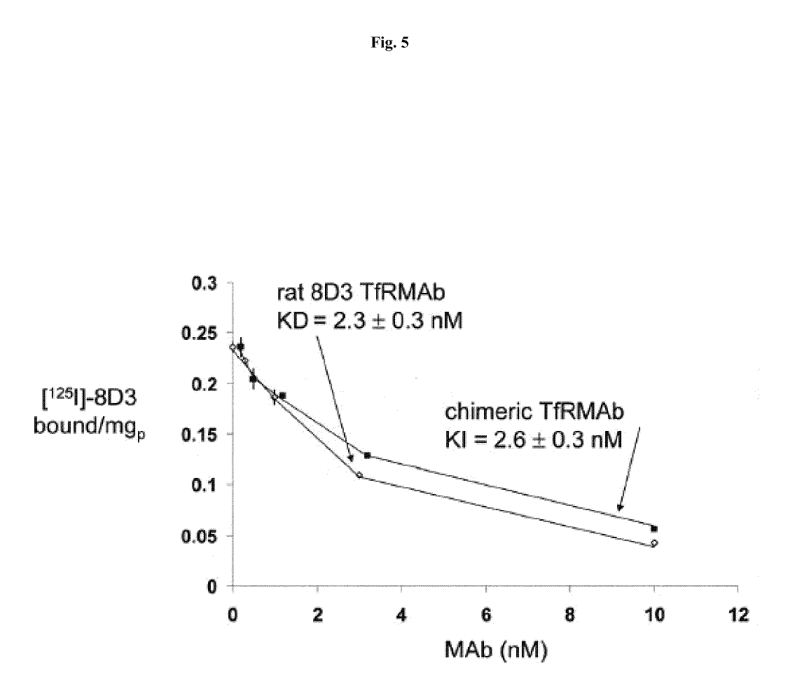

Mouse TfR radio-receptor assay. Mouse fibroblasts were plated in 24-well cluster dishes (100,000 cells/well) 24 hours before the radio-receptor assay (RRA) of TfRMAb binding to the mouse TfR. The medium was aspirated, the cells washed with phosphate buffered saline (PBS), and 500 μL was added to each well containing 0.15 μCi/mL of [¹²⁵I]-labeled rat 8D3 MAb, and 0.3 to 100 nM concentrations of either unlabeled rat 8D3 MAb or unlabeled cTfRMAb. Following incubation at 4 C for 120 min, the medium was aspirated, the plates washed with cold PBS containing 1% bovine serum albumin (BSA), and the monolayer was lysed with 0.2 mL/well of 1 N NaOH. An aliquot was removed for protein measurement using the bicinchoninic acid (BCA) assay, and the radioactivity in the lysate was determined with a Beckmann gamma counter. The % bound/mg protein was computed from the radioactivity in the cell lysate and the total radioactivity in the medium. The 8D3 self-inhibition binding data were fit by non-linear regression analysis yield either the KD of 8D3 self-inhibition in the binding assay, or the KI of cross-inhibition by the chimeric TfRMAb or the cTfRMAb fusion protein. The KI of the chimeric TfRMAb binding to the mouse TfR, 2.6±0.3 nM, was not significantly different from the KD of the hybridoma generated 8D3 TfRMAb to the mouse TfR, 2.3±0.3 nM, as shown in FIG. 5.

### Example 2

### Genetic Engineering of Universal Tandem Vector Encoding IgG-Fusion Proteins

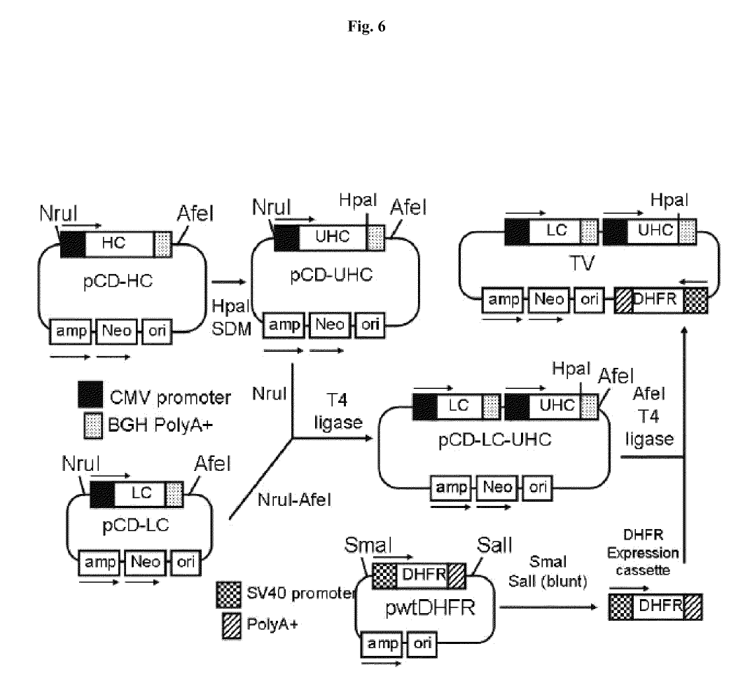

The rat/mouse chimeric anti mTfRMAb protein is comprised of 2 heavy chains (HC) and 2 light chains (LC). Therefore, the host cell may be transfected with both the HC and LC genes. In addition, the host cell can be transfected with a gene that allows for isolation of cell lines with amplification around the transgene insertion site. This is accomplished with selection of cell lines with methotrexate (MTX) following transfection of the host cell with a gene encoding for dihydrofolate reductase (DHFR). Therefore, it is necessary to obtain high production of all 3 genes in a single cell that ultimately produces the Master Cell Bank for manufacturing. In order to insure high expression of all 3 genes, a single piece of DNA, a tandem vector (TV), was engineered, as outlined in FIG. 6. The genetic engineering of the TV for the cTfRMAb protein was completed by successive insertions of both the cTfRMAb LC and the DHFR genes into the cTfRMAb HC expression plasmid (FIG. 6).

In order to produce an expression vector for cTfRMAb fusion proteins, a single HpaI restriction endonuclease (RE) site was introduced by site-directed mutagenesis (SDM) at the stop codon of the mouse IgG1 CH3 region with the ODNs designated pCD-HC-HpaI FWD and REV (SEQ ID NO. 9 and 10, Table 1). This results in a Ser-Ser linker between HC—CH3 and the protein of interest. The HpaI SDM was performed using the pCD-HC plasmid as template to form the vector designated pCD-UHC in FIG. 6. Subsequently, SDM was used to extend the length of the linker to either 3 or 4 serine residues.

The cTfRMAb LC expression cassette was comprised of the cytomegalovirus (CMV) promoter, the TfRMAb LC open reading frame (orf), and the bovine growth hormone (BGH) polyA+ (pA) sequence, and this cassette was released from the pCD-LC expression vector with NruI and AfeI restriction endonuclease digestion and inserted with T4 DNA ligase at the NruI site of the pCD-UHC, located on the 5′-flanking region of the respective CMV sequence (FIG. 6), to form the intermediate vectors designated pCD-LC-UHC (FIG. 6). The engineering of the cTfRMAb TV was later completed by insertion of the DHFR cassette at the AfeI site located on the 3′-flanking region of BGH pA region of the UHC gene in the intermediate pCD-LC-UHC vector (FIG. 6). A mouse wild type (wt) DHFR expression cassette driven by the SV40 promoter and containing the hepatitis B virus transcription terminator was obtained from the pwtDFHR vector (FIG. 6) by digestion with SmaI and SalI. The SalI end was filled with T4 DNA polymerase and deoxynucleotide triphosphates prior to ligation. All intermediate vectors were treated with alkaline phosphatase to prevent self ligation. An internal HpaI RE site in the LC was mutated by SDM using the ODNs LC-HpaI-mut described in Table 1 (SEQ ID NO. 11 and 12), which insured there was only a single HpaI site within the tandem vector for subsequent insertion of therapeutic genes.

The cTfRMAb TV was subjected to bi-directional DNA sequencing. The expression cassettes encoding the LC gene, the HC gene, and the DHFR gene, in the 5′ to 3′ direction were contained within 6,083 nt (SEQ ID NO. 13). The LC cassette was comprised of 1,866 nt, which included a 831 nt CMV promoter, a 9 nt full Kozak sequence (GCCGCCACC), the 705 nt LC orf, and the 321 nt BGH pA sequence. The LC and HC cassettes were separated by a 23 nt linker. The HC cassette was comprised of 2,428 nt, which included a 714 nt CMV promoter, a 9 nt full Kozak sequence (GCCGCCACC), the 1,384 nt HC orf followed by the HpaI site (GTTAAC), and the 315 nt BGH pA sequence. The DHFR cassette was comprised of 1766 nt, which included a 254 nt SV40 promoter, a 9 nt full Kozak sequence (GCCGCCACC), the 564 nt DHFR orf, and the 939 nt hepatitis B virus (HBV) pA sequence. The HC open reading frame (orf) encoded for a 462 amino acid (AA) cTfRMAb HC (SEQ ID NO. 14). The AA sequence, and domain structure, which included a 19 AA signal peptide, is shown in FIG. 3A. The VH CDR1, CDR2, and CDR3 of the cTfRMAb HC are outlined in FIG. 3A, and correspond to amino acids 45-54, 69-85, and 118-126 of SEQ ID NO. 14, respectively. The LC orf encoded for a 234 AA cTfRMAb LC (SEQ ID NO. 15). The AA sequence, and domain structure, which included a 20 AA signal peptide, is shown in FIG. 3B. The VL CDR1, CDR2, and CDR3 of the cTfRMAb LC are outlined in FIG. 3B, and correspond to amino acids 44-54, 70-76, and 109-117 of SEQ ID NO. 15, respectively. The DHFR orf encoded for a 187 AA murine DHFR.

### Example 3

### cTfRMAb-GDNF Fusion Protein

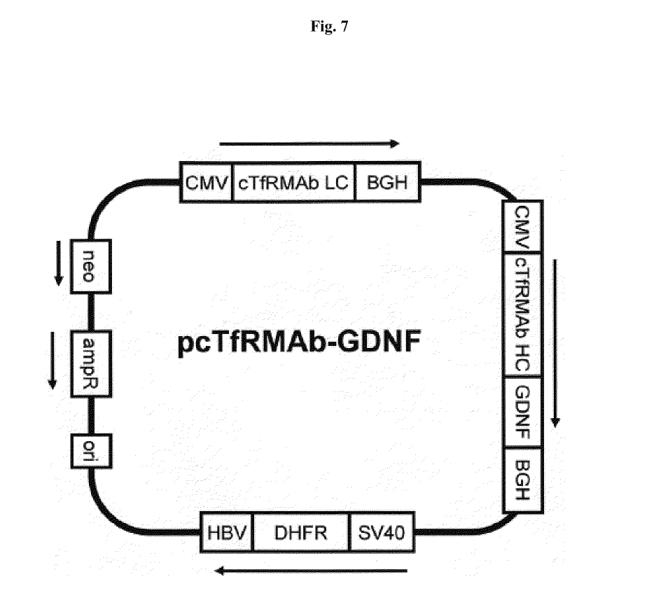

Glial derived neurotrophic factor (GDNF) is a potent neurotrophin for parts of the brain that degenerate in Parkinson's disease (PD). In addition, GDNF could be used to treat motor neuron disease, stroke, alcohol addiction, or drug addiction. Since GDNF does not cross the BBB, GDNF must be re-engineered to cross the human BBB. However, the human BBB delivery systems such as the TfRMAb, are not biologically active in the mouse. It is useful to have a surrogate GDNF-Trojan horse fusion protein that is active in the mouse to enable testing of activity and toxicity in a mouse model of a therapeutic protein-Trojan horse fusion protein that is being developed as a human neurotherapeutic. This would be possible if a cTfRMAb-GDNF fusion protein could be engineered and expressed such that the new fusion protein had dual receptor specificity and bound both the mouse TfR and the human GDNF receptor (GFR) α1 with high affinity. To test whether this was possible, the cDNA encoding mature human GDNF was produced by PCR, and fused to the 3′-end of the cTfRMAb HC gene to produce the HC-GDNF fusion gene, and this was accomplished by insertion of the GDNF cDNA into the HpaI site of the tandem vector (FIG. 6). The HC-GDNF fusion gene, the cTfRMAb LC gene, and the gene encoding murine DHFR were all placed on a new tandem vector, called pcTfRMAb-GDNF (FIG. 7).

The part of the pcTfRMAb-GDNF encompassing the 3 expression cassettes encoding the LC, the HC-GDNF fusion gene, and the DHFR gene, was analyzed by DNA sequencing and spans 6,490 nucleotides (SEQ ID NO. 16). The HC-GDNF fusion protein is comprised of 597 AA (SEQ ID NO. 17), which includes a 19 AA signal peptide. The HC fusion protein without the signal peptide is comprised of 578 AA, and has a predicted MW of 64,018 Da, and a pI of 8.18; the 578 AA includes the 118 AA VH from the rat 8D3 TfRMAb, the 323 AA mouse IgG1 C-region, a 3 AA linker, and the 134 AA human mature GDNF. The LC of the cTfRMAb-GDNF fusion protein is identical to the LC of the cTfRMAb (SEQ ID NO. 15).

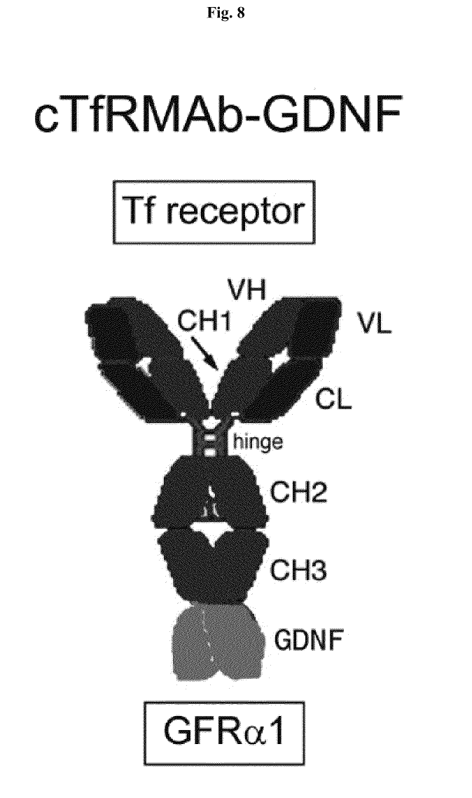

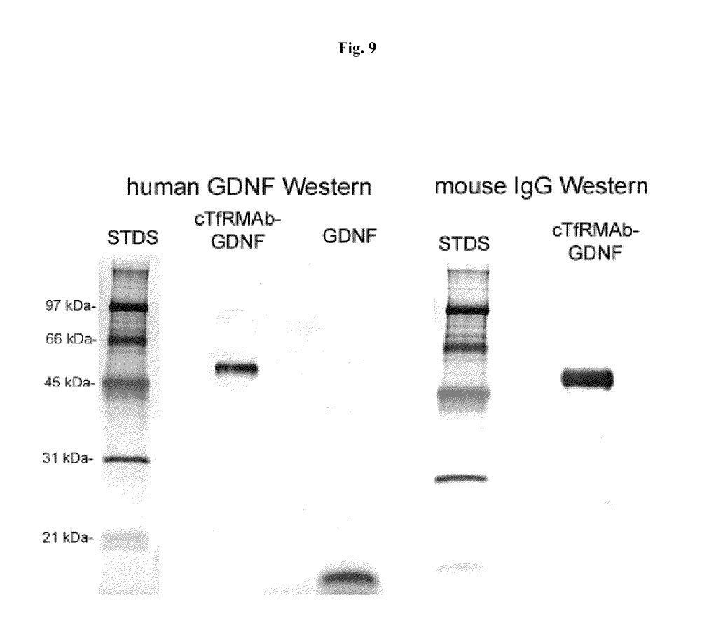

The cTfRMAb-GDNF fusion protein was expressed in COS cells following lipofection of these cells with the pcTfRMAb-GDNF, and the cTfRMAb-GDNF fusion protein was purified from the conditioned serum free medium with protein G affinity chromatography. The TfRMAb-GDNF fusion protein is a hetero-tetrameric molecule, and the structure is shown in FIG. 8. The fusion protein is comprised of 2 light chains and 2 fusion heavy chains. Western blotting of the purified cTfRMAb-GDNF fusion protein with primary antibodies to both mouse IgG and human GDNF showed equal reactivity of these antibodies with the cTfRMAb-GDNF fusion protein (FIG. 9). The anti-GDNF antibody reacts with both the GDNF monomer and the HC of the fusion protein following SDS-PAGE (FIG. 9, left panel). The anti-mouse IgG antibody also reacts with fusion protein heavy chain (FIG. 9, right panel).

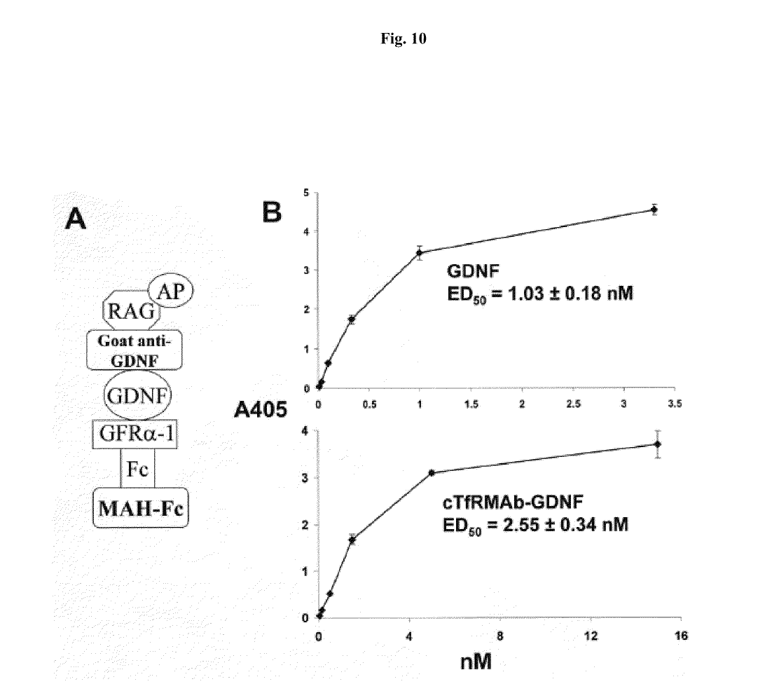

The cTfRMAb-GDNF fusion protein is a bi-functional molecule that binds 2 receptors (FIG. 8): (i) the brain capillary endothelial mouse TfR to cause RMT across the mouse BBB in vivo, and (ii) the GFRα1 on neurons, to cause therapeutic actions in brain behind the BBB. The bi-functionality of the cTfRMAb-GDNF fusion protein was tested with binding assays for both the human GFRα1 and the mouse TfR. The design of the GFRα1 binding assay is shown in FIG. 10A. A mouse anti-human Fc (MAH-Fc) is plated in ELISA wells and captures a fusion protein of human Fc and the human GFRα1 extracellular domain (ECD). The cTfRMAb-GDNF fusion protein, or mature human GDNF, is then added, and these molecules bind to the GFRα1 ECD in proportion to the affinity for this receptor. The binding of the GDNF or the cTfRMAb-GDNF fusion protein is then detected with a goat anti-GDNF antibody, and a rabbit anti-goat (RAG) IgG-alkaline phosphatase (AP) conjugate (FIG. 10A). Human mature 134 amino acid GDNF binds to the GFRα1 with a ED50 of 1.03±0.18 nM (FIG. 10B, top panel), and the cTfRMAb-GDNF fusion protein also binds with high affinity with an ED50 of 2.55±0.34 nM (FIG. 10B, bottom panel). This result shows that the GDNF retains high affinity for its cognate receptor, GFRα1, despite being fused to the carboxyl terminus of the cTfRMAb heavy chain, as shown in FIG. 8.

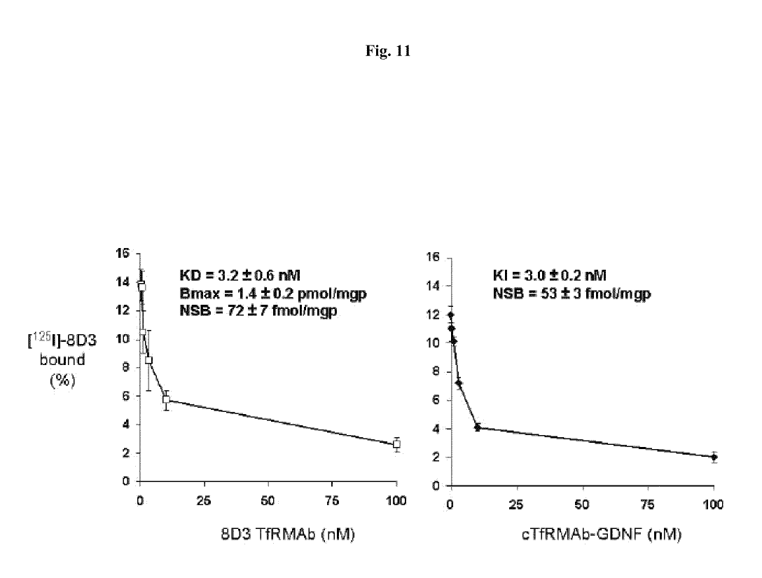

Mouse fibroblasts were used as the source of the mouse TfR, and the ¹²⁵I-labeled rat 8D3 TfRMAb was used as the binding ligand. Unlabeled concentrations of either the 8D3 rat TfRMAb or the cTfRMAb-GDNF fusion protein caused displacement of the [¹²⁵]-8D3 MAb from the TfR. Non-linear regression analysis of the binding isotherms showed the KD of 8D3 binding to the mouse TfR was 3.2±0.3 nM with a Bmax of 1.4±0.2 μmol/mg protein, and a non-specific binding (NSB) of 72±7 fmol/mg protein (FIG. 11, left panel). The KI of the cTfRMAb-GDNF fusion protein inhibition of [¹²⁵]-8D3 TfRMAb binding to the rat TfR was 3.0±0.2 nM (FIG. 11, right panel). Therefore, there is no change in affinity of the cTfRMAb-GDNF fusion protein for the mouse TfR despite fusion of the cTfRMAb to the GDNF protein.

The findings reported in FIGS. 7-11 demonstrate that it is possible to engineer and express a novel fusion protein wherein the amino terminus of human GDNF is fused to the carboxyl terminus of the heavy chain of the cTfRMAb, and that this new fusion protein is bi-functional. The cTfRMAb-GDNF fusion protein binds the mouse TfR with high affinity, to enable transport across the mouse BBB, and binds the human GFRα1 with high affinity to induce pharmacologic effects in the brain behind the BBB.

### Example 4

### cTfRMAb-Avidin Fusion Protein

Short interfering RNA (siRNA) molecules induce RNA interference (RNAi) and are potential new therapeutics for brain diseases, such as brain cancer, brain infection, or polyglutamine disorders such as Huntington's disease. However, siRNAs do not cross the BBB. One strategy for siRNA delivery across the BBB is the combined use of a BBB Trojan horse and avidin-biotin technology. In this approach, the siRNA is mono-biotinylated in parallel with the production of a Trojan horse-avidin fusion protein (Pardridge, (2008), Bioconj. Chem., 19: 1327-1338). The TfRMAb-avidin fusion protein is not biologically active in rodents, and there is no known Trojan horse-avidin fusion protein that is active in the mouse.

To test whether it was possible to produce a cTfRMAb-avidin fusion protein, the cDNA encoding mature avidin was produced by PCR and fused to the 3′-end of the cTfRMAb HC gene to produce the HC-avidin fusion gene, and this was accomplished by insertion of the avidin cDNA into the HpaI site of the tandem vector (FIG. 6). The HC-avidin fusion gene, the cTfRMAb LC gene, and the gene encoding DHFR were all placed on a new tandem vector, called pcTfRMAb-avidin. The part of the pcTfRMAb-avidin tandem vector encompassing the 3 expression cassettes encoding the light chain (LC), the heavy chain (HC)-avidin fusion gene, and the DHFR gene was analyzed by DNA sequencing and spans 6,475 nucleotides (SEQ ID NO. 18). The HC-avidin fusion protein, including the 19 AA signal peptide, is comprised of 592 AA (SEQ ID NO. 19). Without the signal peptide, the HC-avidin fusion protein is comprised of 573 AA, has a predicted MW of 63,375 Da, and a pI of 7.64; the 573 AA include the 118 AA VH from the rat 8D3 TfRMAb, the 323 AA mouse IgG1 C-region, a 4 AA linker, and the 128 AA avidin. The LC of the cTfRMAb-GDNF fusion protein is identical to the LC of the cTfRMAb (SEQ ID NO. 15).

The cTfRMAb-avidin fusion protein was expressed in COS cells following lipofection of these cells with the pcTfRMAb-avidin, and the cTfRMAb-avidin fusion protein was purified from the conditioned serum free medium with protein G affinity chromatography. The cTfRMAb-avidin fusion protein is a hetero-tetrameric molecule, similar to that shown for the cTfRMAb-GDNF fusion protein in FIG. 8. The fusion protein is comprised of 2 light chains and 2 fusion heavy chains. Western blotting of the purified cTfRMAb-GDNF fusion protein with primary antibodies to both mouse IgG and avidin show equal reactivity of these antibodies with the cTfRMAb-avidin fusion protein. The anti-avidin antibody reacts with both the avidin monomer and the HC of the fusion protein following SDS-PAGE. The anti-mouse IgG antibody also reacts with fusion protein heavy chain. The cTfRMAb-GDNF fusion protein is a bi-functional molecule that binds (i) the brain capillary endothelial TfR to cause RMT across the BBB in vivo, and (ii) biotin, to capture biotinylated ligands such as siRNA. To test for binding to the mouse TfR, mouse fibroblasts were used as the source of the mouse TfR, and the ¹²⁵I-labeled rat 8D3 TfRMAb was used as the binding ligand. Unlabeled concentrations of either the 8D3 rat TfRMAb or the cTfRMAb-avidin fusion protein caused displacement of the [¹²⁵]-8D3 MAb from the TfR. Non-linear regression analysis of the binding isotherms showed the KD of 8D3 binding to the mouse TfR was 5.0±0.6 nM; the KI of the cTfRMAb-avidin fusion protein inhibition of [¹²⁵]-8D3 TfRMAb binding to the rat TfR was 4.6±0.5 nM (FIG. 12). Therefore, there is no change in affinity of cTfRMAb binding to the TfR despite fusion of avidin to the cTfRMAb heavy chain.

### Example 5

### cTfRMAb-Single Chain Fv Fusion Protein

Most therapeutic proteins are either monomers, or homo-dimers, such as GDNF. Fusion of the GDNF monomer to the HC of the cTfRMAb, as illustrated in FIG. 8, restores the native dimeric configuration of the GDNF homo-dimer. In the case of therapeutic antibodies, these are generally hetero-dimeric or hetero-tetrameric structures, comprised of separate heavy and light chains. The HC-LC hetero-dimer of a therapeutic antibody can be converted to a single polypeptide by joining the HC and LC polypeptides together with a linker polypeptide to form a single chain Fv (ScFv) antibody. Fusion of the ScFv antibody to the carboxyl terminus of the HC of the cTfRMAb, such as done previously for GDNF (Example 3) and avidin (Example 4), enables the engineering of a fusion antibody comprised of 2 separate antibody molecules: (a) the cTfRMAb, to mediate transport across the BBB of the mouse, and (b) the therapeutic MAb, comprised of the ScFv, which exerts the therapeutic effect in brain behind the BBB.

A model ScFv antibody used is a ScFv against the amino terminal portion of the Aβ amyloid polypeptide of Alzheimer's disease (AD). To test whether it was possible to produce a cTfRMAb-ScFv fusion protein, the cDNA encoding the anti-Aβ ScFv was fused to the 3′-end of the cTfRMAb HC gene to produce the HC-ScFv fusion gene, and this was accomplished by insertion of the ScFv cDNA into the HpaI site of the tandem vector (FIG. 6). The HC-ScFv fusion gene, the cTfRMAb LC gene, and the gene encoding DHFR were all placed on a new tandem vector, called pcTfRMAb-ScFv. The part of the pcTfRMAb-ScFv tandem vector encompassing the 3 expression cassettes encoding the light chain (LC), the heavy chain (HC)-ScFv fusion gene, and the DHFR gene was analyzed by DNA sequencing and spans 6,820 nucleotides (SEQ ID NO. 20). The HC-ScFv fusion protein, including the signal peptide, is comprised of 707 AA (SEQ ID NO. 21). The HC-ScFv fusion protein, minus its signal peptide, is comprised of 688 AA, has a predicted MW of 75,738 Da, and a pI of 7.01; the 688 AA include the 118 AA VH from the rat 8D3 TfRMAb, the 323 AA mouse IgG1 C-region, a 3 AA linker, and the 244 AA ScFv. The LC of the cTfRMAb-ScFv fusion protein is identical to the LC of the cTfRMAb (SEQ ID NO. 15). The VH CDR1, CDR2, and CDR3 of the anti-Aβ ScFv are amino acids 489-498, 513-529, and 562-566 of SEQ ID NO:21, respectively. The VL CDR1, CDR2, and CDR3 of the anti-Aβ ScFv are amino acids 618-633, 649-655, and 688-696 of SEQ ID NO. 21, respectively.

The pcTfRMAb-ScFv tandem vector was used to electroporate Chinese hamster ovary (CHO) cells for permanent transfection of the cells, followed by dilutional cloning of a line expressing and secreting the cTfRMAb-ScFv fusion protein. An ELISA specific for mouse IgG was used to demonstrate secretion of the mouse fusion protein by the transfected CHO cells.

### Example 6

### cTfRMAb In Vivo Pharmacokinetics and Brain Uptake in the Mouse

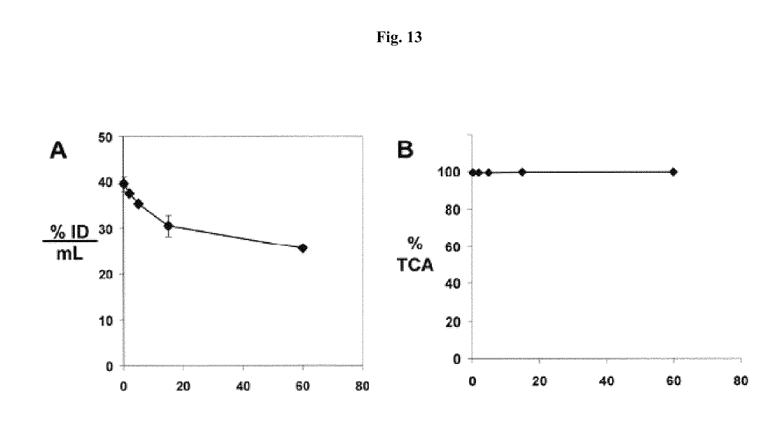

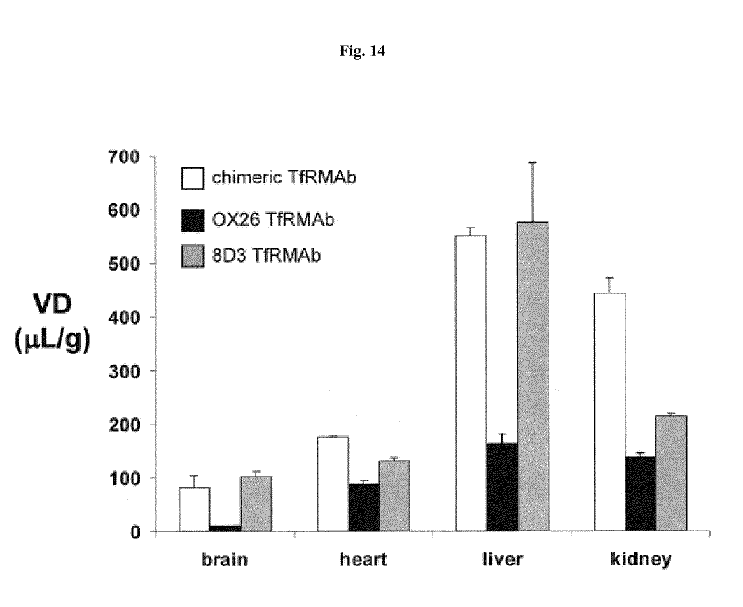

The high binding of the cTfRMAb, or the cTfRMAb fusion proteins, to the TfR, as demonstrated in FIGS. 5, 11, and 12, are predictive of rapid transport across the mouse BBB via RMT on the endogenous mouse TfR. This was verified by measurement of the brain uptake of the cTfRMAb in the mouse in vivo. Adult male BALB/c mice were anesthetized with ketamine/xylazine and injected intravenously with 5 uCi/mouse of [¹²⁵I]-cTfRMAb. Blood was sampled from the common carotid artery at 0.5, 2, 5, 15, and 60 min, and the mouse was euthanized at 60 min for removal of brain, liver, kidney, and heart. The radioactivity was determined in the organ, CPM/g, and in plasma, CPM/uL. Samples of plasma were analyzed by trichloroacetic acid (TCA) precipitation. The organ volume of distribution (VD) was computed from the ratio of CPM/gram to CPM/uL of 60 min organ and plasma radioactivity, respectively. The plasma radioactivity, A(t), was expressed as a % of injected dose (ID)/mL, and was fit to a 2 exponential equation: A(t)=A¹e-^{klt}+A²e-^{k2t}, where Aⁿ and kⁿ are the intercepts and slopes of the exponential decay. The pharmacokinetics parameters were calculated from A¹, A², k¹, and k². The data were fit to both a single and dual exponential decay curve and the residual sum of squares was lowest with the dual exponential function. The [¹²⁵I]-cTfRMAb was cleared from blood at the rate shown in FIG. 13A. The [¹²⁵I]-cTfRMAb was highly stable in vivo as the plasma radioactivity that was TCA-precipitable was >99% in both the pre-injection sample and the 60 min plasma sample (FIG. 13B). The plasma decay curve (FIG. 13A) was subjected to a pharmacokinetics (PK) analysis using a bi-exponential model and the intercepts and slopes are given in Table 2. The calculated PK parameters, including the mean residence time (MRT), the central volume of distribution (Vc), the steady state volume of distribution (Vss), the area under the concentration curve (AUC) at 60 min [AUC(60 min)] and at steady state (AUCss), and the plasma clearance (Cl) were computed and are given in Table 2. The 60 min organ uptake of the [¹²⁵I]-cTfRMAb, expressed as a volume of distribution (VD), was measured for brain, liver, kidney, and heart, and these values are given in FIG. 14, in comparison with VD values in the mouse for the rat hybridoma generated [¹²⁵I]-8D3 TfRMAb, and the mouse hybridoma generated [¹²⁵]-OX26 TfRMAb. The OX26 antibody is a mouse MAb against the rat TfR, and is not active in the mouse (Lee et al, (2000), J. Pharmacol. Exp. Ther, 292: 1048-1052. The chimeric TfRMAb replicates the biological activity of the rat 8D3 MAb in vivo in the mouse (FIG. 14). The chimeric TfRMAb is removed from plasma with a clearance rate of 0.47±0.13 mL/min/kg (Table 2), and this rate is comparable to the clearance of the 8D3 MAb in mice, 0.24±0.03 mL/min/kg (Lee et al supra). The brain VD of the chimeric TfRMAb is comparable to the brain VD for the 8D3 MAb in the mouse, whereas the brain VD of the murine OX26 MAb to the rat TfR is very low in the mouse (FIG. 14). The murine OX26 MAb to the rat TfR does not recognize the mouse TfR, is not transported across the mouse BBB (Lee et al supra), and functions as a blood volume marker in the mouse. The blood volume in peripheral organs is much higher than in brain, which is represented by the higher VD of the OX26 MAb in mouse heart, liver, and kidney, as compared to the brain (FIG. 14). The high VD of the chimeric TfRMAb in heart is due mainly to distribution in the high blood volume in that organ, as the VD of the chimeric TfRMAb or the 8D3 TfRMAb in heart is not much higher than the OX26 MAb (FIG. 14). In contrast, the VD in liver of the chimeric TfRMAb or the 8D3 MAb is very high compared to the blood volume as represented by the VD of the OX26 MAb (FIG. 14), which indicates the chimeric TfRMAb and 8D3 antibodies are selectively taken up by the liver TfR. With respect to kidney, the uptake of the chimeric TfRMAb is somewhat higher than the uptake of the 8D3 TfRMAb (FIG. 14). These in vivo studies corroborate the in vitro radio-receptor assays (FIGS. 5, 11, and 12) of binding to the mouse TfR. The combined studies show that the genetically engineered chimeric TfRMAb has the same activity of binding to the mouse TfR in vitro, and the same BBB transport in vivo, as the original rat hybridoma generated 8D3 MAb.

### Example 7

### Variation of Mouse Constant Regions

The domain structure of the HC of the fusion protein, including the complementarity determining regions (CDRs) and framework regions (FR) of the chimeric TfRMAb HC are given in FIG. 3A. The constant region is derived from mouse IgG1, and the amino acid sequence comprising the CH1, hinge, CH2, and CH3 is given in FIG. 3A. In addition, the HC C-region could be derived from the C-region of other mouse IgG isotypes, including mouse IgG2, IgG3, and IgG4. The different C-region isotypes each offer well known advantages or disadvantages pertaining to flexibility around the hinge region, protease sensitivity, activation of complement or binding to the Fc receptor. The domain structure of the LC, including the CDRs and FRs of the chimeric TfRMAb LC, are given in FIG. 3B. The constant region is derived from mouse kappa LC, and the amino acid sequence comprising the mouse kappa constant region is given in FIG. 3B. In addition, the light chain C-region could be derived from the mouse lambda light chain isotype.

### Example 8

### Variation of the Linker Separating the IgG Chain and the Therapeutic Protein

The heavy chain fusion proteins described above were engineered with a linker comprised of either 3 amino acids (Ser-Ser-Ser), or 4 amino acids (Ser-Ser-Ser-Ser) between the IgG heavy chain and the therapeutic protein. In the sequences described in SEQ ID NO. 17 and 21, there is a Ser-Ser-Ser linker at amino acids 461-463. In the sequence described in SEQ ID NO. 19, there is a Ser-Ser-Ser-Ser linker at amino acids 461-464. A variety of other linkers could be used to join the IgG chain and the therapeutic protein, such as a single amino acid or a dipeptide, or an extended linker could be used. For example, an extended Gly/Ser or GS linker, such as a GGGGSGGGGSGGGGS linker (SEQ ID NO:22), designated GS15, could be introduced at the original short linker to form the extended linker SGGGGSGGGGSGGGGSS (SEQ ID NO:23). Or, a variety of other linkers could be substituted for the short or extended amino acid linkers.

TABLE 1
PCR primers for cloning 8D3 VH and VL regions, and
mouse IgG1 heavy chain C-region and mouse kappa
light chain C-region

8D3 VH forward

(SEQ ID NO. 1)

5′-ATCCTCGAGGTTAACTGGTGGAGTCTGGAGGAGG-3′
8D3 VH reverse

(SEQ ID NO. 2)

5′-GGGGGTGTCGTTTTAGCTGAGGAGACAGTG-3′
8D3 VL forward

(SEQ ID NO. 3)

5′-GGTGATATCGT(G/T)CTCAC(C/T)CA(A/G)TCTCCAGCAAT-3′
8D3 VL reverse

(SEQ ID NO. 4)

5′-GGGAAGATGGATCCAGTTGGTGCAGCATCAGC-3′
Mouse IgG1 forward

(SEQ ID NO. 5)

5′-CAGCCGGCCATGGCGCAGGTSCAGCTGCAGSAG-3′
Mouse IgG1 reverse

(SEQ ID NO. 6)

5′-TCATTTACCAGGAGAGTGGGAGAG-3′
Mouse kappa forward

(SEQ ID NO. 7)

5′-AATTTTCAGAAGCACGCGTAGATATCKTGMTSACCCAAWCTCCA-3′
Mouse kappa reverse

(SEQ ID NO. 8)

5′-TCAACACTCTCCCCTGTTGAAGCTC-3′
pCD-HC-HpaI FWD

(SEQ ID NO. 9)

5′-CACTCTCCTGGTAAAAGTTAACCACCACACTGGACT-3′
pCD-HC-HpaI REV

(SEQ ID NO. 10)

5′-AGTCCAGTGTGGTGGTTAACTTTTACCAGGAGAGTG-3′
LC-HpaI-mut FWD

(SEQ ID NO. 11)

5′-CCAGTGAGCAGTTGACATCTGGAGGTGCC-3′
LC-HpaI-mut REV

(SEQ ID NO. 12)

5′-GGCACCTCCAGATGTCAACTGCTCACTGG-3′

The mouse IgG1 reverse primer is complementary to the end of the mouse IgG1 heavy chain C-region (GenBank U65534). The mouse kappa reverse primer is complementary to the end of the mouse kappa light chain C-region (GenBank Z37499). K=G or T; M=A or C; S=G or C; W=A or T.

TABLE 2
Pharmacokinetic parameters of chimeric TfRMAb in the mouse

| Parameter | Units | Value |
|---|---|---|
| A¹ | % ID/mL | 18.4 ± 4.2 |
| A² | % ID/mL | 33.9 ± 2.1 |
| k¹ | min⁻¹ | 0.71 ± 0.37 |
| k² | min⁻¹ | 0.0048 ± 0.0016 |
| t_{1/2}¹ | min | 0.98 ± 0.51 |
| t_{1/2}² | min | 144 ± 48 |
| MRT | min | 208 ± 68 |
| Vc | mL/kg | 64 ± 5 |
| Vss | mL/kg | 97 ± 6 |
| AUC(60 min) | % ID · min/mL | 1794 ± 60 |
| AUCss | % ID · min/mL | 7098 ± 2010 |
| Cl | mL/min/kg | 0.47 ± 0.13 |

MRT = mean residence time;
Vc = plasma volume;
Vss = steady state volume of distribution;
AUC(60 min) = area under the curve first 60 min;
AUCss = steady state AUC;
Cl = clearance from plasma

### Example 9

### Treatment with cTfRMAb-IDUA in a Mouse Model of Mucopolysaccharidosis (MPS) Type I

Homozygous MPS I B6.129^{-Iduatm1Clk/J/Iduatm1Clk/J} mice (Jackson Labs, Bar Harbor, Me.) with a knock-out of the alpha-L-iduronidase gene (see Clarke et al (1997), Hum Mol. Genet., 6(4):503-511) are bred to obtain homozygous mutant offspring. Adult homozygous mutant mice (6-14 weeks) are anesthetized with ketamine/xylazine and injected intravenously with 0.1, 0.5, or 5 mg/kg of purified cTfRMAb (control) or an equimolar dose of a fusion antibody comprising human IDUA (GenBank No. NP_{—}000194) fused at its amino terminus via a three amino acid linker (SSS) to the C-terminal of the cTfRMAb HC (SEQ ID NO:14) of the mouse cTfRMAb described above. After 30 minutes, mice are euthanized and plasma and brain tissue are collected. IDUA activity in plasma and brain tissue homogenate is measured in a fluorometric assay with 4-methylumbelliferyl-α-L-iduronide as described in, e.g., Hartung et al (2004), Mol Ther, 9:866-875. Enzymatic activity is expressed as nmol 4-methylumbelliferone released per mg tissue protein per hour (nmol/mg/h) or per ml plasma per hour (nmol/ml/h).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising a purified chimeric monoclonal antibody against the mouse transferrin receptor.

2. The composition of claim 1, comprising a fusion protein comprising the chimeric monoclonal antibody against the mouse transferrin receptor and a CNS-active polypeptide, wherein the CNS-active polypeptide is covalently linked to a heavy chain or a light chain of the chimeric monoclonal antibody.

3. The composition of claim 2, wherein the antibody and the CNS-active agent each retain an average of at least 10% of their activities, compared to their activities as separate entities.

4. The composition of claim 2, wherein the CNS-active polypeptide comprises the amino acid sequence of a neurotrophin, a single chain Fv antibody, an avidin, or an enzyme.

5. The composition of claim 2, wherein the CNS-active polypeptide is covalently linked at its N-terminus to the C-terminus of the chimeric monoclonal antibody heavy chain or light chain.

6. A method for delivering a therapeutic agent across the BBB in a mouse, comprising administering to the mouse a composition comprising the composition of claim 2.

7. The composition of claim 4, wherein the CNS-active polypeptide is a neurotrophin.

8. The composition of claim 4, wherein the CNS-active polypeptide is avidin.

9. The composition of claim 4, wherein the CNS-active polypeptide is a ScFv.

10. The composition of claim 4, wherein the CNS-active polypeptide is an enzyme.

11. A nucleic acid encoding a heavy chain immunoglobulin or a light chain immunoglobulin of a monoclonal antibody against the mouse transferrin receptor.

12. The nucleic acid of claim 11, wherein the nucleic acid encodes the heavy chain immunoglobulin and the light chain immunoglobulin.

13. The nucleic acid of claim 11, further encoding a CNS-active polypeptide fused in frame to the encoded heavy chain immunoglobulin or light chain immunoglobulin.

14. The nucleic acid of claim 13, wherein the encoded CNS-active polypeptide comprises the amino acid sequence of a neurotrophin, a single chain Fv antibody, or an avidin.

15. The nucleic acid of claim 11, wherein the nucleic acid hybridizes under medium stringency conditions to a nucleic acid comprising the nucleic acid sequence of any of SEQ ID NOs: 13, 16, 20, or its complement.

16. The nucleic acid of claim 11, wherein the nucleic acid hybridizes under medium stringency conditions to a nucleic acid encoding a polypeptide comprising the amino acid sequence of any of SEQ ID NOs:14, 15, 17, 19, 21, or to the complement of the nucleic acid sequence encoding the polypeptide.

17. A recombinant mouse comprising a chimeric monoclonal antibody against the mouse transferrin receptor.

18. The recombinant mouse of claim 17, comprising a fusion protein comprising the chimeric monoclonal antibody against the mouse transferrin receptor and a CNS-active polypeptide, wherein the CNS-active polypeptide is covalently linked to a heavy chain or a light chain of the chimeric monoclonal antibody.

19. The recombinant mouse of claim 18, wherein the CNS-active polypeptide comprises the amino acid sequence of a neurotrophin, a single chain Fv antibody, or avidin.

20. The recombinant mouse of claim 18, wherein the CNS-active polypeptide is covalently at its N-terminus to the C-terminus of the chimeric monoclonal antibody heavy chain or light chain.

21. The recombinant mouse of claim 19, wherein the neurotrophin comprises an amino acid sequence at least 85% identical to that of a human neurotrophin.


SEQUENCE LISTING (25 sequences)

SEQ ID NO: 1 - DNA, 34 bp, Artificial Sequence
  Description of Artificial Sequence Synthetic primer
atcctcgaggttaactggtggagtctggaggagg

SEQ ID NO: 2 - DNA, 30 bp, Artificial Sequence
  Description of Artificial Sequence Synthetic primer
gggggtgtcgttttagctgaggagacagtg

SEQ ID NO: 3 - DNA, 32 bp, Artificial Sequence
  Description of Artificial Sequence Synthetic primer
ggtgatatcgtkctcacycartctccagcaat

SEQ ID NO: 4 - DNA, 32 bp, Artificial Sequence
  Description of Artificial Sequence Synthetic primer
gggaagatggatccagttggtgcagcatcagc

SEQ ID NO: 5 - DNA, 33 bp, Artificial Sequence
  Description of Artificial Sequence Synthetic primer
cagccggccatggcgcaggtscagctgcagsag

SEQ ID NO: 6 - DNA, 24 bp, Artificial Sequence
  Description of Artificial Sequence Synthetic primer
tcatttaccaggagagtgggagag

SEQ ID NO: 7 - DNA, 44 bp, Artificial Sequence
  Description of Artificial Sequence Synthetic primer
aattttcagaagcacgcgtagatatcktgmtsacccaawctcca

SEQ ID NO: 8 - DNA, 25 bp, Artificial Sequence
  Description of Artificial Sequence Synthetic primer
tcaacactctcccctgttgaagctc

SEQ ID NO: 9 - DNA, 36 bp, Artificial Sequence
  Description of Artificial Sequence Synthetic primer
cactctcctggtaaaagttaaccaccacactggact

SEQ ID NO: 10 - DNA, 36 bp, Artificial Sequence
  Description of Artificial Sequence Synthetic primer
agtccagtgtggtggttaacttttaccaggagagtg

SEQ ID NO: 11 - DNA, 29 bp, Artificial Sequence
  Description of Artificial Sequence Synthetic primer
ccagtgagcagttgacatctggaggtgcc

SEQ ID NO: 12 - DNA, 29 bp, Artificial Sequence
  Description of Artificial Sequence Synthetic primer
ggcacctccagatgtcaactgctcactgg

SEQ ID NO: 13 - DNA, 6083 bp, Artificial Sequence
  Description of Artificial Sequence Synthetic polynucleotide
taagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcaggccgccaccatggagacccccgcccagctgctgttcctgttgctgctttggcttccagatactaccggcgatatccagatgactcaatctccagcctccctgtctgcatctctggaagaaattgtcaccatcacatgccaggcaagccaggacattggaaattggttggcatggtatcagcagaaaccagggaaatctcctcagctcctgatctatggtgcaaccagcttggcagatggggtcccatcaaggttcagcggcagtagatctggcacacagttttctcttaagatcagcagagtacaggttgaagatattggaatctattactgtctacaggcttataatactccgtggacgttcggtggaggcaccaagctggaattgaaacgggctgatgctgcaccaactgtatccatcttcccaccatccagtgagcagttgacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttgagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggagccgccaccatggactggacctggagggtgttctgcctgcttgcagtggcccccggagcccacagcgaggttcaactggtggagtctggaggaggcttagtacagcctggaaattccctgacactctcatgtgttgcctctggattcactttcagtaactatggcatgcactggattcgccaggctccaaagaaggggctggaatggatagcaatgatttattatgatagtagtaaaatgaactacgcagacaccgtgaagggtcgattcaccatttccagagacaattctaagaacaccctgtacctggaaatgaacagtttgagatctgaggacacagccatgtattactgtgcggtaccgacaagccactatgttgtggatgtctggggtcaaggagtttcagtcactgtctcctcagctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggaagtagttaaccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcgggaggtaccgagctcttacgcgtgctagctcgagatctgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgattctagaagccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagc

SEQ ID NO: 14 - protein, 462 aa, Artificial Sequence
  Description of Artificial Sequence Synthetic polypeptide
MDWTWRVFCLLAVAPGAHSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGSS

SEQ ID NO: 15 - protein, 234 aa, Artificial Sequence
  Description of Artificial Sequence Synthetic polypeptide
METPAQLLFLLLLWLPDTTGDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 16 - DNA, 6490 bp, Artificial Sequence
  Description of Artificial Sequence Synthetic polynucleotide
taagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcaggccgccaccatggagacccccgcccagctgctgttcctgttgctgctttggcttccagatactaccggcgatatccagatgactcaatctccagcctccctgtctgcatctctggaagaaattgtcaccatcacatgccaggcaagccaggacattggaaattggttggcatggtatcagcagaaaccagggaaatctcctcagctcctgatctatggtgcaaccagcttggcagatggggtcccatcaaggttcagcggcagtagatctggcacacagttttctcttaagatcagcagagtacaggttgaagatattggaatctattactgtctacaggcttataatactccgtggacgttcggtggaggcaccaagctggaattgaaacgggctgatgctgcaccaactgtatccatcttcccaccatccagtgagcagttgacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttgagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggagccgccaccatggactggacctggagggtgttctgcctgcttgcagtggcccccggagcccacagcgaggttcaactggtggagtctggaggaggcttagtacagcctggaaattccctgacactctcatgtgttgcctctggattcactttcagtaactatggcatgcactggattcgccaggctccaaagaaggggctggaatggatagcaatgatttattatgatagtagtaaaatgaactacgcagacaccgtgaagggtcgattcaccatttccagagacaattctaagaacaccctgtacctggaaatgaacagtttgagatctgaggacacagccatgtattactgtgcggtaccgacaagccactatgttgtggatgtctggggtcaaggagtttcagtcactgtctcctcagctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggaagtagttcatcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaaaccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcgggaggtaccgagctcttacgcgtgctagctcgagatctgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgattctagaagccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagc

SEQ ID NO: 17 - protein, 597 aa, Artificial Sequence
  Description of Artificial Sequence Synthetic polypeptide
MDWTWRVFCLLAVAPGAHSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGSSSSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 18 - DNA, 6475 bp, Artificial Sequence
  Description of Artificial Sequence Synthetic polynucleotide
taagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcaggccgccaccatggagacccccgcccagctgctgttcctgttgctgctttggcttccagatactaccggcgatatccagatgactcaatctccagcctccctgtctgcatctctggaagaaattgtcaccatcacatgccaggcaagccaggacattggaaattggttggcatggtatcagcagaaaccagggaaatctcctcagctcctgatctatggtgcaaccagcttggcagatggggtcccatcaaggttcagcggcagtagatctggcacacagttttctcttaagatcagcagagtacaggttgaagatattggaatctattactgtctacaggcttataatactccgtggacgttcggtggaggcaccaagctggaattgaaacgggctgatgctgcaccaactgtatccatcttcccaccatccagtgagcagttgacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttgagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggagccgccaccatggactggacctggagggtgttctgcctgcttgcagtggcccccggagcccacagcgaggttcaactggtggagtctggaggaggcttagtacagcctggaaattccctgacactctcatgtgttgcctctggattcactttcagtaactatggcatgcactggattcgccaggctccaaagaaggggctggaatggatagcaatgatttattatgatagtagtaaaatgaactacgcagacaccgtgaagggtcgattcaccatttccagagacaattctaagaacaccctgtacctggaaatgaacagtttgagatctgaggacacagccatgtattactgtgcggtaccgacaagccactatgttgtggatgtctggggtcaaggagtttcagtcactgtctcctcagctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggaagtagttcgtctgccagaaagtgctcgctgactgggaaatggaccaacgatctgggctccaacatgaccatcggggctgtgaacagcagaggtgaattcacaggcacctacatcacagccgtaacagccacatcaaatgagatcaaagagtcaccactgcatgggacacaaaacaccatcaacaagaggacccagcccacctttggcttcaccgtcaattggaagttttcagagtccaccactgtcttcacgggccagtgcttcatagacaggaatgggaaggaggtcctgaagaccatgtggctgctgcggtcaagtgttaatgacattggtgatgactggaaagctaccagggtcggcatcaacatcttcactcgcctgcgcacacagaaggagtgaaaccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcgggaggtaccgagctcttacgcgtgctagctcgagatctgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgattctagaagccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagc

SEQ ID NO: 19 - protein, 592 aa, Artificial Sequence
  Description of Artificial Sequence Synthetic polypeptide
MDWTWRVFCLLAVAPGAHSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGSSSSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 20 - DNA, 6820 bp, Artificial Sequence
  Description of Artificial Sequence Synthetic polynucleotide
taagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcaggccgccaccatggagacccccgcccagctgctgttcctgttgctgctttggcttccagatactaccggcgatatccagatgactcaatctccagcctccctgtctgcatctctggaagaaattgtcaccatcacatgccaggcaagccaggacattggaaattggttggcatggtatcagcagaaaccagggaaatctcctcagctcctgatctatggtgcaaccagcttggcagatggggtcccatcaaggttcagcggcagtagatctggcacacagttttctcttaagatcagcagagtacaggttgaagatattggaatctattactgtctacaggcttataatactccgtggacgttcggtggaggcaccaagctggaattgaaacgggctgatgctgcaccaactgtatccatcttcccaccatccagtgagcagttgacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttgagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggagccgccaccatggactggacctggagggtgttctgcctgcttgcagtggcccccggagcccacagcgaggttcaactggtggagtctggaggaggcttagtacagcctggaaattccctgacactctcatgtgttgcctctggattcactttcagtaactatggcatgcactggattcgccaggctccaaagaaggggctggaatggatagcaatgatttattatgatagtagtaaaatgaactacgcagacaccgtgaagggtcgattcaccatttccagagacaattctaagaacaccctgtacctggaaatgaacagtttgagatctgaggacacagccatgtattactgtgcggtaccgacaagccactatgttgtggatgtctggggtcaaggagtttcagtcactgtctcctcagctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggaagtagttcacaggtccagctgcagcagtctggggctgaactggtgaagcctggggctacagtgaagttgtcctgcaaggcttctggctacagtttcaacagtcactatatatattgggtgaagcagaggcctggacaaggccttgagtggattggagagattaatcctagcaatggtgctatgaacttcaatgagaagttcaagaataaggccacactgactgtagacaaatcctccagcacagcttacatgcagctcagcagcctgacatctgaggactctgcggtctattattgtgtaagggaccctacgtcttactggggccaggggactctggtcactgtctctgcagccaaaacgacacccaagcttgaagaaggtgaattttcagaagcacgcgtagatgtcgtgatgacccaaactccactctccctgcctgtcagtcttggagatcaagcctccatctcttgcagatctagtcagagccttgtacacagttatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaacggtaaaaccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcgggaggtaccgagctcttacgcgtgctagctcgagatctgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgattctagaagccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagc

SEQ ID NO: 21 - protein, 707 aa, Artificial Sequence
  Description of Artificial Sequence Synthetic polypeptide
MDWTWRVFCLLAVAPGAHSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGSSSQVQLQQSGAELVKPGATVKLSCKASGYSFNSHYIYWVKQRPGQGLEWIGEINPSNGAMNFNEKFKNKATLTVDKSSSTAYMQLSSLTSEDSAVYYCVRDPTSYWGQGTLVTVSAAKTTPKLEEGEFSEARVDVVMTQTPLSLPVSLGDQASISCRSSQSLVHSYGNTYLHWYLQKPGQSPKLLIYKVSNRFSGVPDRFSGSGSGTDFTLKISRVEAEDLGVYFCSQSTHVPYTFGGGTKLEIKR

SEQ ID NO: 22 - protein, 15 aa, Artificial Sequence
  Description of Artificial Sequence Synthetic peptide
GGGGSGGGGSGGGGS

SEQ ID NO: 23 - protein, 16 aa, Artificial Sequence
  Description of Artificial Sequence Synthetic peptide
SGGGGSGGGGSGGGGS

SEQ ID NO: 24 - protein, 4 aa, Artificial Sequence
  Description of Artificial Sequence Synthetic peptide
SSSS

SEQ ID NO: 25 - protein, 461 aa, Artificial Sequence
  Description of Artificial Sequence Synthetic polypeptide
MDWTWRVFCLLAVAPGAHSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK